(19) United States
(12) Patent Application Publication
(10) Pub. No.: US 2020/0003772 A1
(43) Pub. Date: Jan. 2, 2020

(54) NON-INVASIVE BRAIN INJURY DIAGNOSTIC DEVICE

(71) Applicant: MEDICORTEX FINLAND OY, Turku (FI)

(72) Inventors: Adrian HAREL, Turku (FI); Lasse VÄLIMAA, Lieto (FI)

(21) Appl. No.: 16/486,286

(22) PCT Filed: Feb. 5, 2018
(86) PCT No.: PCT/IB2018/050698

§ 371 Date: Aug. 15, 2019

Related U.S. Application Data
(60) Provisional application No. 62/461,277, filed on Feb. 21, 2017.

(51) Int. Cl. G01N 33/543; A61B 10/00; G01N 33/53; A61B 5/00; G01N 33/68

(52) U.S. Cl. CPC G01N 33/54386; A61B 10/0051; A61B 10/007; G01N 2400/10; A61B 5/4064; G01N 33/6896; G01N 2400/02; G01N 33/5308

(57) ABSTRACT

Disclosed is a device for conducting a non-invasive diagnostic test in a subject suspected of suffering brain injury. The device for diagnosing a brain injury in a subject includes a probe of a porous matrix, an indicator formulation disposed on the porous matrix and includes at least one lectin and/or antibody capable of selectively binding to a glycan-based biomarker indicative of brain injury in a sample, and a visually detectable label; and a handle in communication with the probe, wherein at least one of the lectin and/or antibody and/or the visually detectable label is immobilized in and/or on a detection zone in the porous matrix, and the visually detectable label develops a color intensity level and becomes visible upon a binding event of the glycan-based biomarker to the lectin and/or antibody. Also provided is a method for using the device described below and methods for producing the same.

### FIELD AND BACKGROUND OF THE INVENTION

The present invention, in some embodiments thereof, relates to a diagnostic device and method and, more particularly, but not exclusively, to a portable, user-initiated visual assay device and method for diagnosing brain injury.

Traumatic brain injury (TBI) is the leading cause of central nervous system impairment in these days, with more than 1.7 million individuals suffering annually from TBI in the US alone. According to the CDC, the highest incidence of TBI occurs among children 0-4 years old, adolescents 15-19 years old, and adults over 65 years of age. Despite the broad range of the population affected, TBI is still under-served and remains an unexplored pathological condition.

Traditionally, TBI has been acutely diagnosed and classified by neurological examinations, such as Glasgow Coma Scale (GCS). However, the use of the GCS as a diagnostic tool is subject to a number of important limitations. Recent research has provided evidence that the use of sedative drugs precluded accurate GCS assessment during the first 24 hours. Further challenges to diagnosis are presented by the evolving nature of some brain lesions, which can lead to further neurological impairment. In addition, neurological responses after TBI can vary over time for reasons unrelated to the injury. Still further challenges include the trauma subject's possible unconsciousness or inability to communicate.

Neuroimaging techniques, such as x-ray, CT scanning and MRI, are used to provide information on injury magnitude and location, and are not influenced by the aforementioned disadvantages. However, CT scanning has low sensitivity to diffuse brain damage, and availability and utility of MRI is limited. MRI is also very impractical to perform if subjects are physiologically unstable, and can lead to inaccurate diagnoses in military injuries in which metal fragments are common.

Mild and moderate TBI represent more than 90% of TBI injuries; this injury range represents the greatest challenges to accurate acute diagnosis and outcome prediction. Unlike severe TBI, there is no universally recognized neurologic assessment scale such as the GCS, and many cases of mild TBI are classified as subclinical brain injury (SCI). The widespread recognition of inadequate approaches to diagnose mild TBI suggests the need for significant improvement in the diagnosis and classification of TBI, such as the use of biomarkers to supplement functional and imaging-based assessments. These biomarkers can be altered gene expression, protein or lipid metabolites, or a combination of these changes after traumatic brain injury, reflecting the initial insult (the primary injury) and the evolution of a cascade of secondary damage (the secondary injury). In particular, subclinical brain injury status or SCI could be diagnosed with a biomarker analysis.

As with many injuries, increased serum levels of cytokines and chemokines have been noted post-TBI and, as such, have been proposed as potential surrogate markers for TBI outcome. However, to date, there are no approved biomarkers for the diagnosis or prognosis of TBI. This is because of several obstacles to the development of reliable blood biomarkers of TBI. For instance, the blood-brain barrier (BBB) hinders the assessment of biochemical changes in the brain by use of blood biomarkers in mild TBI, although impaired BBB integrity, as seen in severe TBI, can increase the levels of brain-derived proteins in the blood. Nevertheless, owing to their dilution in the much larger plasma volume, biomarkers that are highly expressed within the central nervous system exist at very low concentrations in blood. Moreover, some potential biomarkers undergo proteolytic degradation in the blood, and their levels might be affected by clearance from blood via the liver or kidney. As a consequence, reliable blood biomarkers have been extremely difficult to identify.

WO/2016/166419 by the present assignee and one of the present inventors, which is incorporated by reference in its entirety, discloses glycan-based biomarkers for the diagnosis and prognosis of brain damage, such as traumatic brain injury (TBI), subclinical brain injury (SCI) and acquired brain injury (ABI). The glycan-based biomarker protocol disclosed therein may be used as an end point in clinical trials and in other diagnostic tests to determine, qualify, and/or assess brain injury status, for example, to diagnose brain injury, in an individual, subject or patient. As part of the diagnosis afforded by the glycan-based biomarker disclosed therein, brain injury status can include determination of a subject's subclinical brain injury status or SCI status, for example, to diagnose SCI, in an individual, subject or patient (conscious or not).

Nonetheless, most diagnostic methodologies, such as the provisions of WO/2016/166419, call for sample extraction, preparation and assaying, which is carried out by healthcare specialists using special reagents and equipment, as well as analytical protocols that require specific professional training. Unfortunately, the ever-growing strain on the healthcare system, the increased prevalence of common injuries and diseases, and the substantial delay in treatment caused by instrument-access queues and remote testing, stands in the way of utilizing technologies such as provided in WO/2016/166419.

Historic obstacles to point-of-care devices include manufacturing challenges, ease-of-use limitations, and government regulations. Some of these obstacles have been reduced through advances in technology and recognition by governments and other regulatory bodies of the importance of point-of-care testing. However, important considerations, including ease-of-use and accuracy, still render point-of-care tests unsuitable for many healthcare facilities and domestic settings, and more so for particular medical conditions, such as brain injury.

The use of reagent-impregnated test strips in specific binding assays, such as immunoassays, has previously been proposed. In such procedures a sample is applied to one portion of the test strip and is allowed to permeate through the strip material, usually with the aid of an eluting solvent such as water or an appropriate buffer solution. In so doing, the sample progresses into or through a detection zones in the test strip wherein a specific binding reagent for an analyte suspected of being in the sample is immobilized. Analyte present in the sample can therefore become bound within the detection zone. The extent to which the analyte becomes bound in that zone can be determined with the aid of labelled reagents which can also be incorporated in the test strip or applied thereto subsequently. Examples of prior proposals utilizing these principles are given in U.S. Pat. Nos. 5,602,040, 8,802,427, 8,927,262, 8,999,728, 9,052,311 and 9,151,754, GB 1589234, EP 0225054, EP 0183442 and EP 0186799.

Additional prior art documents include, U.S. Pat. Nos. 7,993,283 and 9,366,674, and U.S. Patent Application Publication No. 20160257989.

### SUMMARY OF THE INVENTION

The present invention provides, inter alia, a device for conducting a non-invasive analysis of a bodily fluid, such as saliva or urine, to determine the presence and the level of a certain glycan-based biomarkers that are indicative of brain injury, that are carried by the bodily fluid. The device includes an indicator formulation capable of changing color in response to exposure to the biomarkers to provide a visual indication of the presence and the level of the biomarkers carried by the bodily fluid. The device comprises a porous matrix substrate for establishing a high void volume within the carrier substrate, and an indicator formulation carried by the carrier substrate. The indicator formulation includes a chromogen agent (a visually detectable label) and a biomarker-specific agent selected from a variety of agents responsive to levels of any one of a plurality of different glycan-based biomarkers that are indicative of brain injury. In addition, the present invention provides a method for using the device described below.

Thus, one object of the present invention is to provide a test device which is readily usable by an unskilled person and which preferably merely requires that some portion of the device is contacted with the sample (e.g., saliva or urine), and thereafter no actions or minimal simple actions are required by the user before a diagnostic or an analytical result can be observed. Preferably the diagnostic/analytical result is observable within a matter of minutes following sample application, e.g., ten minutes or less.

According to an aspect of some embodiments of the present invention, there is provided a device for diagnosing a brain injury in a subject, which includes:

a probe, the probe includes a porous matrix; and

an indicator formulation disposed in and/or on the porous matrix and includes at least one glycan-based biomarker binding reagent for selectively binding to a glycan-based biomarker in a sample, and a first visually detectable label;

wherein:

at least one of the glycan-based biomarker binding reagent and/or the first visually detectable label is immobilized in and/or on a detection zone in the porous matrix;

the glycan-based biomarker is indicative of brain injury;

the first visually detectable label develops a color and becomes visible upon a binding event of the glycan-based biomarker to the glycan-based biomarker binding reagent; and

the binding event is effected by contacting the sample with the probe.

In some embodiments, the glycan-based biomarker binding reagent is a lectin and/or an antibody.

In some embodiments, the first visually detectable label is attached to the glycan-based biomarker binding reagent.

In some embodiments, the probe further includes a control formulation, the control formulation includes a control binding reagent and a second visually detectable label, the control binding reagent binds at least one of the glycan-based biomarker binding reagent, a glycan and any complex thereof, and the second visually detectable label becomes visible upon a binding event of the control binding reagent to the glycan-based biomarker binding reagent, the glycan and/or the complex thereof, wherein the control binding reagent and/or the second visually detectable label is immobilized in and/or on a control zone in the porous matrix.

In some embodiments, a change in an intensity level of the color is proportional to a concentration level of the glycan-based biomarker in the sample.

In some embodiments, the device further includes a semi-permeable layer disposed over the probe, the layer is permeable to aqueous media and aqueous solutes therein, and is impermeable to particles larger than 0.05 μm.

In some embodiments, the device further includes a handle in communication with the probe.

In some embodiments, the handle includes a tube in direct communication with the probe on a proximal end thereof, and open on a distal end thereof, the tube is for transporting the sample and/or a solution from an external source to or from the probe (a portal).

In some embodiments, the device further includes a frame having an opening, and the probe is housed within the opening in the plane of the frame, and the frame is mounted on the handle.

In some embodiments, the frame includes a color intensity gauge, the gauge includes a plurality of areas arranged radially around the opening, each of the areas is having a color intensity level representing a concentration level of the glycan-based biomarker in the sample, for a visual comparison of a color intensity level in the probe with a color intensity level in one of the areas in the gauge, thereby providing a direct visual determination of a concentration level of the glycan-based biomarker in the sample.

In some embodiments, the device presented herein is essentially as presented in FIG. 1.

In some embodiments, the device presented herein is essentially as presented in FIGS. 2A-C.

In some embodiments, the device presented herein is essentially as presented in FIG. 3.

In some embodiments, the device presented herein is essentially as presented in FIG. 4.

In some embodiments, the device presented herein is essentially as presented in FIGS. 6A-D.

In some embodiments, the sample is urine, and the handle is a tube configured for effecting the contacting.

In some embodiments, the sample is saliva, and the device is sized and shaped for insertion into the subject's mouth for effecting the contacting.

According an aspect of some embodiments of the present invention, there is provided a device for diagnosing a brain injury in a subject, which includes:

a flat round probe, the probe includes a porous matrix;

an indicator formulation disposed in and/or on a detection zone in the porous matrix and includes at least one glycan-based biomarker binding reagent for selectively binding to a glycan-based biomarker in a sample, and a first visually detectable label;

a control formulation disposed in and/or on a control zone in the porous matrix and includes a control binding reagent and a second visually detectable label; and

a handle in communication with the probe,

wherein:

the glycan-based biomarker is indicative of brain injury;

at least one of the glycan-based biomarker binding reagent and/or the first visually detectable label is immobilized in and/or on the detection zone;

the first visually detectable label develops a color and becomes visible upon a binding event of the glycan-based biomarker to the glycan-based biomarker binding reagent;

the control binding reagent binds at least one of the glycan-based biomarker binding reagent, a glycan and any complex thereof;

the control binding reagent and/or the second visually detectable label is immobilized in and/or on the control zone;

the second visually detectable label becomes visible upon a binding event of the control binding reagent to the glycan-based biomarker binding reagent, the glycan and/or the complex thereof; and

the binding event is effected by contacting the sample with the probe.

In some embodiments, the handle includes a tube in direct communication with the probe on a proximal end thereof, and open on a distal end thereof, the tube is for transporting the sample and/or a solution from an external source to the probe.

In some embodiments, the handle is configured in a shape selected from the group consisting of a syringe tip fitting/adaptor, a stretchable and elastic fitting/adaptor, a screw threaded fitting/adaptor, a piercing needle tip fitting/adaptor, a septum membrane and a butterfly needle fitting/adaptor.

In some embodiments, the handle is configured in a shape of a syringe.

In some embodiments, the control zone and the detection zone are perpendicular to one another and overlap at the center so as to form a cross pattern.

According an aspect of some embodiments of the present invention, there is provided a non-invasive method for diagnosing brain injury in a subject, the method is effected by:

contacting the probe in any of the devices presented herein with the sample;

assessing a visible change in the control zone, if present; and

determining brain injury in a subject according to a color change in the detection zone,

wherein the change in the color is effected by the binding event of the glycan-based biomarker to the glycan-based biomarker binding reagent, and indicative of a brain injury in the subject.

In some embodiments, the sample is saliva or urine.

In some embodiments, contacting the probe with the sample is effected by inserting the device to the mouth of the subject and wetting the probe with saliva.

In some embodiments, contacting the probe with the sample is effected by wetting the probe with urine of the subject.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic illustration of an exemplary “strip” shaped device, according to some embodiments of the present invention, wherein device 10, having detection zone 11 and handle 12, is dipped into sample 13 not having a glycan-based biomarker, which lead to no coloring of wet detection zone 15, but when dipped into sample 14 having a glycan-based biomarker, wet detection zone 16 changes color;

FIG. 2A presents a schematic diagram of lollipop device, wherein probe 20 is having mobile labeled antibody or lectin (analyte-specific binding reagent) 21 disposed thereon, and when a saliva or urine sample containing glycan-based biomarker (analyte) 22 is contacted with probe 20, mobile labeled antibody/lectin-biomarker adduct 23 is formed;

FIG. 2B presents a schematic diagram of the device presented in FIG. 2A, wherein some of mobile labeled antibody or lectin 21 was at or has migrated to horizontal control zone 24, in which nonspecific antibody or lectin 25 is immobilized on the porous matrix of probe 20, and the binding event is made visible by the label on mobile labeled antibody or lectin 21, now immobilized and concentrated in control zone 24 as visibly detectable control complex 26, indicating that the device is functioning properly;

FIG. 2C presents a schematic diagram of the device presented in FIGS. 2A-B, wherein some of mobile labeled antibody/lectin-biomarker adduct 23 was at or has migrated to perpendicular detection zone 27, in which biomarker-specific antibody/lectin 28 is immobilized on the porous matrix of probe 20, and the binding event is made visible by the label on mobile labeled antibody/lectin-biomarker adduct 23, now immobilized and concentrated in detection zone 27 as visibly detectable diagnostic complex 29, indicating that the sample contacted with the device contains glycan-based biomarkers indicative of brain injury;

FIG. 3 presents a schematic illustration of a device, according to some embodiments of the present invention, wherein device 30 is having probe 31 comprising porous matrix 32 in which control zone 33 and detection zone 34 form a “plus” sign and handle 35 is a rigid hollow tube designed connect to the tip of generic syringe 36 and transfer the liquid sample to probe 31;

FIG. 4 presents a schematic illustration of a device, according to some embodiments of the present invention, wherein device 40 includes probe 41 that comprises indicator formulation 42, and housed within frame 44, mounted on handle 43, whereas the plurality of areas 45a-g are arranged radially around the opening in frame 44, and control zone 46 is positioned at the center of probe 41;

FIG. 5 presents a schematic illustration of a device, according to some embodiments of the present invention, wherein device 50 includes probe 51 that comprises indicator formulation 52 and control zone 56 is positioned at the center of probe 51, mounted on handle 53, and separate gauge 54 having a plurality of areas 55a-g; and

FIGS. 6A-D present schematic illustrations of some embodiments of the present invention, wherein FIG. 6A shows a device having probe 61 in direct communication with handle portal 62 and additional portals 63 branching off from handle portal 62, FIG. 6B shows a device having probe 61 and two portals 64 in direct communication with probe 61, FIG. 6C shows a device having portal 64 in direct communication with probe 61 and additional portals 63 branching off from handle portal 62, and FIG. 6D shows a device having probe 61 in direct communication with handle reservoir 65 in the form of a syringe that is secured from accidental or premature ejection of its content by plunger stopper 66 as part of a kit and protective sheath (such as metallic or plastic pouch or container) 67 that can also serve as a sample dipping container as part of a kit.

### DESCRIPTION OF SOME SPECIFIC EMBODIMENTS OF THE INVENTION

The present invention, in some embodiments thereof, relates to a diagnostic device and method and, more particularly, but not exclusively, to a portable, user-initiated visual assay device and method for diagnosing brain injury.

The principles and operation of the present invention may be better understood with reference to the figures and accompanying descriptions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

As discussed hereinabove, given the great strain on the healthcare work force, the increased prevalence of many common injuries, diseases and the substantial delay in treatment caused by remote testing, the present inventors have recognized the need for self-monitoring non-invasive means for diagnosing brain injury. The present inventors have contemplated a user-friendly non-invasive mode for brain injury diagnosis, which allows the use of an inexpensive apparatus suitable for more widespread off-clinic use and acceptance that enables greater convenience in carrying about and use in testing and provides a simplified visual mode for monitoring test results. The present inventors have also contemplated an off-the-shelf product that enables the economical manufacture and distribution of relatively low-cost, reliable diagnostic device that can be used by non-professionals in educational, sports, and other public facilities, as well as homes and workplaces.

While reducing the present invention to practice, the present inventors have envisioned rapid, easy-to-use diagnostic devices and methods to enable efficient and accurate point-of-care (POC) detection of brain injury, which comprising a means for saliva stimulation, a candy-like (lollipop) component that may or may not feature a taste or aroma, a means for visual change activation, and a gauge for visual comparison of the results.

### Portable Non-Invasive Visual Diagnosis Device:

In the context of some embodiments of the present invention, the device for diagnosing brain injury is based on the detecting certain glycan-based biomarkers in a sample, as these are described in details hereinbelow, wherein the sample is obtained by non-invasive means, such as saliva and urine, and the indication of positive or negative diagnosis of a brain injury is obtained without need for special machinery and/or processes, and can be carried out by a layman. Nonetheless, it is noted herein that use of the provisions of the present invention are not limited to samples extracted by non-invasive methods, meaning that the device and methods provided herein can be used to diagnose brain injury by sampling blood, plasma, spinal fluid and the like.

The present inventors have considered that some colorimetric and enzymatic reporter systems useful in detecting glycan-based biomarkers are used as solutions and the formed colors spread by diffusion, which makes them less suitable in portable off-clinic POC (at home) device, designed for highlighting a pattern on a membrane or any solid support. In addition, concentrated acids, heating or harmful chemicals are used in several colorimetric reactions known in the art for detecting proteins and saccharides. Thus, in some embodiments, the detection of glycan-based biomarkers is effected by non-toxic, non-hazardous and generally safe reagents that evoke a chromatic reaction that is visually perceptible, which requires no further equipment or processing to be developed and observed by a non-professional user.

One object of the present invention is to provide a test device which is readily usable by an unskilled person and which preferably merely requires that some portion of the device is contacted with the sample (e.g., saliva or urine), and thereafter no further actions, or only minimal simple actions, such as shaking, mixing, pushing a plunger etc., are required by the user before a diagnostic or an analytical result can be observed. Preferably the diagnostic/analytical result is observable within a matter of minutes following sample application, e.g., ten minutes or less. Such devices can be provided as kits suitable for home use, comprising a plurality (e.g., more than one) of devices individually wrapped in moisture impervious wrapping and packaged together with appropriate instructions to the user.

Some embodiments of the present invention are focused on adapting and improving some of the known analyte detection techniques and methodologies, such as those referred to herein, to provide brain injury diagnostic test devices especially suitable for home use which are quick and convenient to use and which require the user to perform as few actions as possible.

Thus, according to an aspect of some embodiments of the present invention, there is provided a device for diagnosing brain injury in a subject. The device includes:

a probe that includes an porous matrix; and

an indicator formulation disposed in and/or on the porous matrix and comprises at least one glycan-based biomarker binding reagent capable of selectively binding to a glycan-based biomarker in a sample, and a first visually detectable label.

In some embodiments, the indicator formulation includes at least one glycan-based biomarker binding reagent capable of selectively binding to a glycan-based biomarker in a liquid sample taken non-invasively from the subject, and a visually detectable label, wherein:

the glycan-based biomarker is indicative of brain injury;

the glycan-based biomarker binding reagent and/or the visually detectable label is immobilized on the porous matrix;

the visually detectable label develops a color and becomes visible upon a binding event of the glycan-based biomarker to the glycan-based biomarker binding reagent; and

the binding event is effected by contacting the sample with the probe.

In some embodiments, the glycan-based biomarker binding reagent is a lectin, a galectin, or an antibody. Herein and throughout, unless stated otherwise, the term “glycan-based biomarker binding reagent”, refers to any one of the antibodies, lectins, galectins or other molecules which has been identified as capable of selectively bind to a glycan-based biomarker. In the context of embodiments of the present invention, the glycan-based biomarker is indicative of brain injury in a subject. It is also noted that unless stated otherwise, a reference to an antibody as glycan-based biomarker binding reagent, is meant to encompass lectins, galectins or other molecules which has been identified as capable of selectively bind to a glycan-based biomarker; a reference to a lectin as glycan-based biomarker binding reagent, is meant to encompass antibodies, galectins or other molecules which has been identified as capable of selectively bind to a glycan-based biomarker; and a reference to a galectin as glycan-based biomarker binding reagent, is meant to encompass lectins, antibodies or other molecules which has been identified as capable of selectively bind to a glycan-based biomarker

In some embodiments, a dye/colorant/chromogen forms a colored complex or changes its color in the presence of a glycan-base biomarker (chemical glycan assays). In such embodiments, the detection of glycan-based biomarkers is not necessarily based on binding thereof to a specific affinity binding reagent, but rather on the mere presence of the biomarker and its effect on other factors in the probe. For example, a reaction cascade is initiated by the presence of the biomarker, which causes a change in color in the probe. The reaction may or may not include enzymes. In some embodiments, an enzyme specific for the glycan-based biomarker starts a conversion reaction in the presence of the biomarker. The enzymatic reaction is coupled to a dye/colorant/chromogen which develops color or change it color (enzymatic activity). Such detection mechanism also does not require immobilization of any element in the indicator formulation, and the color change may be effected throughout the probe. Such approach is particularly suitable for the strip device embodiments described hereinbelow.

Some embodiments of the present invention include diagnostic test devices removably encased in a wrapping material or a casing container constructed of moisture-impervious solid material.

The device of the present invention comprises a probe that includes a dry porous carrier (matrix), referred to herein as a “porous matrix”, which designed to carry the indicator formulation, and to be soaked with a liquid test sample that is applied to the probe. The probe may further be sectioned into zones, such as a detection zone and a control zone, as these are described hereinbelow.

In some embodiments, the device of the present invention further includes a handle in communication with the probe, designed for handling the probe for sample contacting and the like.

In some embodiments, the handle includes or is a tube, which is in direct communication with the porous matrix of the probe on the proximal end thereof (the end that is connected to the probe). The distal end of the tube handle is open to receive a liquid sample such that the tube can transport the sample from an external source to said probe. In such embodiments of the present invention, the handle is used also as an inlet and/or outlet portal to infuse liquids and reagents in solution into and/or out of the probe. The liquid can be a sample and/or a standard analyte solution and/or an indicator formulation reagent and/or a washing liquid, and any combination thereof. The handle and the distal end thereof can be shaped as a syringe tip fitting/adaptor, or be stretchable and elastic for fitting any tip of the external source of the sample, or be a screw threaded tip, a piercing needle tip, a septum membrane, a butterfly needle adaptor, and have any shape designed to connect to an external source of a liquid sample.

In some embodiments, the purpose of the tube, in addition to introducing the sample, is to deliver additional reagents in solution to the probe to start/enhance/stop the reaction, if needed. The additional solution may carry an element that assists in the color development, and or supplement the indicator formulation with a detection element, if needed.

The term “portal”, as used in the context of some embodiments of the present invention, refers to an element of the device, which is designed as an inlet and/or outlet for infusing or retracting liquids and reagents in solution into and/or out of the probe. In some embodiments, the device includes more than one portal, as described above, for letting into the probe any one or more of a sample and/or a standard analyte solution and/or an indicator formulation reagent and/or a washing liquid, and any combination thereof. In such embodiments the handle can have a multiple inlets and outlets portals, or be connected to a manifold of inlets and outlets, or the probe can be in communication with more than one portal regardless of a handle.

In some embodiments, the device is equipped with at least one portal to which a reservoir is attached. The reservoir may be in the form of a piston/plunger and cylinder/barrel) combination (e.g., a syringe), wherein the plunger is retracted and the barrel is the reservoir. In some embodiments, the reservoir can be pre-filled with a liquid that is used in the diagnosis process, and can be, for example, a standard analyte solution and/or an indicator formulation reagent and/or a washing liquid, and any combination thereof.

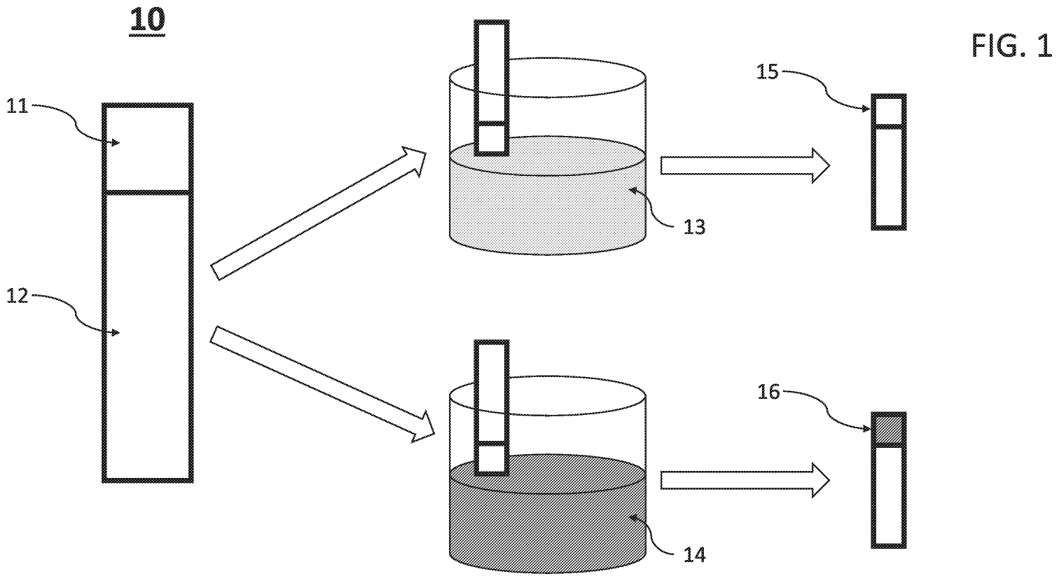

In some embodiments, the device has a shape of a strip, namely an elongated flat thin object, wherein one end thereof or a mid-section thereof, serves as a probe, and one or two tips or ends thereof serve as a handle. An illustration of an exemplary strip-shaped device is discussed hereinbelow and presented in FIG. 1.

In some embodiments, the probe is further coated or tightly wrapped with a layer of a semi-permeable material. The material of the layer is selected to be permeable to aqueous media and aqueous solutes therein, and to be impermeable to particles larger than a certain threshold, such as 0.01 μm, 0.02 μm, 0.03 μm, 0.04 μm, 0.05 μm, 0.1 μm, 0.2 μm, 0.3 μm, 0.4 μm or 0.5 μm. This layer provides a user interface and a mean to prevent passage of mobile elements in the probe to pass to the user when contacted to absorb a liquid sample, (e.g., when the probe is inserted into the mouth to be soaked with saliva).

Suitable semi-permeable membranes, such as the type of biological or synthetic, polymeric membrane that will allow certain molecules or ions to pass through it by diffusion, or occasionally by more specialized processes of facilitated diffusion, passive transport or active transport. Suitable semi-permeable membranes, composed of either regenerated cellulose or cellulose esters (e.g., cellulose acetate) are manufactured through distinct processes of modifying and cross-linking cellulose fibers derived from wood pulp or cotton fibers to form films with differing properties and pore sizes. Variations in the manufacturing process significantly change the properties and pores sizes of the film. Cellulose-based membranes are also suitable. Glycerol is frequently added as a humectant to prevent cracking during drying and to help maintain the desired pore structure. Regenerated cellulose membranes are very hydrophilic and hydrate rapidly when introduced to water. Due to their additional crosslinking, regenerated cellulose membranes have better chemical compatibility and heat stability than membranes made from cellulose esters. Regenerated cellulose membranes are also more resistant to organic solvents and to the weak or dilute acids and bases that are commonly used in protein and molecular biology applications.

### Porous Matrix:

The probe may be constructed from a porous matrix “backed” with a support material, e.g. with a plastic sheet, to increase its handling strength. This can be manufactured easily by forming a thin layer of the porous matrix on a sheet of backing material. Alternatively, a pre-formed sheet of porous matrix can be tightly sandwiched between two supporting sheets of solid material, e.g., plastic sheets.

The porous matrix, which is the sample receiving member, can be made from any bibulous, porous or fibrous material capable of absorbing liquid rapidly. The porosity of the material can be unidirectional (i.e., with pores or fibers running wholly or predominantly parallel to an axis of the member) or multidirectional (omnidirectional, so that the member has an amorphous sponge-like structure). Porous plastics material, such as polypropylene, polyethylene (preferably of very high molecular weight), polyvinylidene flouride, ethylene vinylacetate, acrylonitrile and polytetrafluoroethylene can be used. It can be advantageous to pre-treat the member with a surface-active agent during manufacture, as this can reduce any inherent hydrophobicity in the member and therefore enhance its ability to take up and deliver a moist sample rapidly and efficiently. Porous sample receiving members can also be made from paper or other cellulosic materials, such as nitrocellulose. Materials that are widely used in the nibs of so-called fiber tipped pens are particularly suitable and such materials can be shaped or extruded in a variety of lengths and cross-sections appropriate in the context of the invention. Preferably the material comprising the porous receiving member should be chosen such that the porous member can be saturated with aqueous liquid within a matter of seconds. Preferably the material remains robust when moist.

In some embodiments of the invention, the porous matrix is selected from the family of nitrocellulose materials. This family has some advantage over conventional synthetic or cellulose materials, such as paper, because it has a natural ability to bind proteins without requiring prior sensitization. Specific binding reagents, such as lectins and immunoglobulins (antibodies), can be applied directly to nitrocellulose and immobilized thereon. No chemical treatment is required which might interfere with the essential specific binding activity of the reagent. Unused binding sites on the nitrocellulose can thereafter be blocked using simple materials, such as polyvinylalcohol. Moreover, nitrocellulose is generally safe, non-toxic and readily available in a range of pore sizes and this facilitates the selection of a carrier material to suit particularly requirements such as sample flow rate.

Preferably the porous matrix has a pore size of at least one micron. Preferably the porous matrix has a pore size not greater than about 20 microns. In some embodiments, the average pore size of the porous matrix ranges 1-10, 1-20, 1-30, 1-40 or 1-50 microns.

In some embodiments of the present invention, the probe includes a solid phase porous matrix which is linked to a porous receiving member to which the liquid sample can be applied and from which the sample can permeate into the porous matrix. Preferably, the porous matrix is contained within a moisture-impermeable casing or housing and the porous receiving member, with which the porous matrix is linked, extends out of the housing and can act as a means for permitting a liquid sample to enter the housing and permeate the porous solid phase material. The housing should be provided with means, e.g., appropriately placed apertures, which enable the second zone of the porous solid phase material (carrying the immobilized unlabeled specific binding reagent) to be observable from outside the housing so that the result of the assay can be observed. If desired, the housing may also be provided with further means which enable a further zone of the porous solid phase material to be observed from outside the housing and which further zone incorporates control reagents which enable an indication to be given as to whether the assay procedure has been completed. Preferably the housing is provided with a removable cap or shroud which can protect the protruding porous receiving member during storage before use. If desired, the cap or shroud can be replaced over the protruding porous receiving member, after sample application, while the assay procedure is being performed. Optionally, the labeled reagent can be incorporated elsewhere within the device.

Blocking of unused binding sites in the porous matrix can be achieved by treatment with protein (e.g. bovine serum albumin or milk protein), or with polyvinylalcohol or ethanolamine, or any combination of these agents, for example. The mobile reagent(s) can then be dispensed onto the dry matrix and will become mobile in the carrier when in the moist state. Between each of these various process steps (sensitization, application of unlabeled reagent, blocking and application of the labeled reagent), the porous matrix should be dried.

The various reagents can be applied to the probe in a variety of ways. Various “printing” techniques have previously been proposed for application of liquid reagents to porous matrices, e.g. micro-syringes, pens using metered pumps, direct printing and inkjet printing, and any of these techniques can be used in the present context. To facilitate manufacture, the matrix can be treated with the reagents and then subdivided into smaller portions, e.g., small narrow strips each embodying the required reagent-containing zones, to provide a plurality of identical carrier units.

### Indicator Formulation:

The porous matrix contains an indicator formulation, which is a general term that is used to refer to a system that comprises a number of reagents and labels, some may be attached to one-another, some may be immobilized on the matrix and some are freely mobile therein in the moist state, and all are selected to bind, label and immobilize an analyte of interest found in the sample, or to form a colored complex with the analyte, or to change color in the presence of the analyte, which are not necessarily affinity-pair binding-based assays. The indicator formulation thus includes specific binding reagents for an analyte, wherein the specific binding reagents (glycan-based biomarker binding reagents) are typically lectins and/or antibodies, and the analyte is one or more glycan-based biomarkers, at least some of which are indicative of a brain injury. In some embodiments, the lectin and the antibody are specific to the same glycan-based biomarker(s), which are indicative of a brain injury in the subject being tested.

The indicator formulation further includes a labeling agent, referred to herein as a “visually detectable label”. In some embodiments, the lectin and/or antibody is labelled with the visually detectable label, namely the visually detectable label is chemically attached to the lectin and/or to the antibody. The specific binding reagents and the visually detectable labels are selected such that upon a binding event of the specific binding reagents to the glycan-based biomarker(s) (or upon contacting a glycan-based biomarker, when using diffusible dyes and colors), the visually detectable label develops a color having a color intensity level, which has not been visible prior to the binding event, and thus becomes visible thereby making the binding even visibly distinguishable. In some embodiments, wherein a control for non-specific binding, or a “timer” mechanisms are used, there may be two or more different kids on visually detectable labels employed in the device, and in such cases, the visually detectable label used for visualization of specific binding, is referred to herein as a first visually detectable label. In these cases, a visually detectable label used for the “control” or “timer” mechanisms is referred to herein as a second visually detectable label. In some cases, the first and second visually detectable labels are identical.

In the context of embodiments of the present invention, the term “visible” refer to a visual signal that can be detected by the naked eye (visible light which a human eye can perceive), without the use of additional machinery or processes. In the context of embodiments of the present invention, a visible signal is a change in a color of a certain object or an area thereon, relative to the color that has been characteristic to the object or area prior to the change. A change can also be assessed in comparison to the background of the object or area, and in comparison to the surrounding of the object or area.

In some embodiments, the labeled or unlabeled lectin and/or antibody is permanently immobilized in a detection zone in/on the porous matrix and is therefore not mobile in the moist state (when the probe is soaked with the liquid sample). The detection zone can be the entire area of the probe, or a predetermined area thereof, which can have a visibly recognized shape, such as a dot, a circle, a bar, a square and the like.

In some embodiments, a labeled or unlabeled specific binding reagent is freely mobile within the porous matrix when in the moist state, and another labeled or unlabeled specific binding reagent for the same analyte is permanently immobilized in the detection zone on the porous matrix and is therefore not mobile in the moist state, and the relative positioning of the labelled reagent and detection zone being such that liquid sample containing the analyte applied to the probe of the device can pick up labelled reagent and thereafter permeate into the detection zone, wherein a three-membered binding event causes a color change in the detection zone. The color change may also be a change in the color intensity level.

In one example, the porous matrix contains an indicator formulation that comprises a labelled specific binding reagent for an analyte which is freely mobile within the porous matrix when in the moist state, and an unlabeled specific binding reagent for the same analyte is permanently immobilized in the detection zone on the porous matrix and is therefore not mobile in the moist state. In such configuration, typically referred to as a “sandwich” configuration, the analyte and the freely mobile labeled binding reagent bind to one-another, thereby specifically labeling the analyte indirectly with the visually detectable label, and the formed labeled mobile adduct is picked-up by the immobilized unlabeled specific binding reagent to form a sandwich that is positioned permanently at the detection zone, where the color develops due to accumulation of the visually detectable label at high concentration, relative to other areas in the probe not having an immobilized reagent, if those are present.

The immobilized specific binding reagent in the detection zone is preferably a highly specific antibody, lectin or galectin. In some embodiments, the immobilized species is a monoclonal antibody. In the embodiment of the invention involving the sandwich reaction, the labeled reagent is a lectin or also preferably a highly specific antibody, and more preferably a monoclonal antibody.

The basic elements in the foregoing can be utilized, according to some embodiments of the present invention, in a “competition” assay mode, wherein the analyte in the sample (glycan-based biomarker) is in competition with a labeled version thereof for the limited number of binding sites (immobilized specific binding reagents) on the probe. In such a “competition” assay, the detectible signal can be a decrease in the color intensity level, or a change in color in cases where the background color becomes more visible when the labeled version of the analyte depletes from the detection zone.

Thus, another embodiment of the invention is a device for use in an assay for an analyte, incorporating a porous solid phase material carrying in a first zone a labelled reagent which is retained in the first zone while the porous material is in the dry state but is free to migrate through the porous material when the porous matrix is moistened, for example by the application of an aqueous liquid sample suspected of containing the analyte, the porous material carrying in a second zone, which is spatially distinct from the first zone, an unlabeled specific binding reagent having specificity for the analyte, and which is capable of participating with the labelled reagent in either a “sandwich” or a “competition” reaction, the unlabeled specific binding reagent being firmly immobilized on the porous material such that it is not free to migrate when the porous material is in the moist state.

The invention also provides an analytical method in which a device as set forth in the foregoing is contacted with an aqueous liquid sample suspected of containing the analyte, such that the sample permeates by capillary action through the solid phase porous matrix via the first zone into the second zone and the labelled reagent migrates therewith from the first zone to the second zone, the presence of analyte in the sample being determined by observing the extent (if any) to which the labeled reagent becomes bound in the second zone.

In another embodiment of the invention, the labeled reagent is a specific binding partner for the analyte. The labeled reagent, the analyte (if present) and the immobilized unlabeled specific binding reagent cooperate together in a “sandwich” reaction. This results in the labeled reagent being bound in the second zone if analyte is present in the sample. The two binding reagents have specificities for different epitopes on the analyte.

In another embodiment of the invention, the labeled reagent is either the analyte itself which has been conjugated with a label, or is an analyte analogue, i.e., a chemical entity having the identical specific binding characteristics as the analyte, and which similarly has been conjugated with a label. In the latter case, it is preferable that the properties of the analyte analogue which influence its solubility or dispersibility in an aqueous liquid sample and its ability to migrate through the moist solid phase porous matrix should be identical to those of the analyte itself, or at least very closely similar. In this embodiment, the labeled analyte or analyte analogue will migrate through the solid phase porous matrix into the second zone and bind with the immobilized reagent. Any analyte present in the sample will compete with the labelled reagent in this binding “competition” reaction. Such competition will result in a reduction in the amount of labeled reagent binding in the second zone, and a consequent decrease in the intensity of the signal observed in the second zone in comparison with the signal that is observed in the absence of analyte in the sample.

Embodiments of the present invention are meant to encompass any methodology and system for specific labeling and detection of analytes that is useful for visual determination of an analyte in a non-invasive and simple to use as the device presented herein. Particular useful are methodologies and systems for specific labeling and detection of lectins, glycans and antibodies, such as described below; and as provided in the art by, for example, Tao, S. C. et al. [“Lectin microarrays identify cell-specific and functionally significant cell surface glycan markers”, Glycobiology, 2008, 18(10), pp. 761-769], Katrlík, J. et al. [“Glycan and lectin microarrays for glycomics and medicinal applications”, Med Res Rev, 2010, 30(2), pp. 394-418, ISSN: 0198-6325], Hirabayashi, J. et al. [“Lectin-based structural glycomics: A practical approach to complex glycans”, Electrophoresis, 2011, 32(10), pp. 1118-1128], and Hirabayashi, J. et al. [“Lectin microarrays: concept, principle and applications”, Chemical Society Reviews, 2013, 42(10), pp. 4443-4458].

### Visually Detectable Label:

The visually detectable label can be any entity the presence of which can be readily detected. Preferably the label is a direct label, i.e., an entity which, in its natural state, is readily visible either to the naked eye, or with the aid of an optical filter and/or applied stimulation, e.g., UV light to promote fluorescence. For example, minute colored particles, such as dye sols/colloids, metallic sols/colloids (e.g., gold colloid), carbon black particles and nanotubes, and colored latex particles, are suitable in the context of some embodiments of the present invention. Of these options, colored latex particles are most preferred. Concentration of the label into a small zone or volume should give rise to a readily detectable signal, e.g. a strongly-colored area. This can be evaluated by eye, or by instruments if desired.

Indirect labels, such as enzymes, e.g. alkaline phosphatase and horseradish peroxidase, can be used. These labels usually require the addition of one or more developing reagents such as substrates before a visible signal can be detected. Such additional reagents can be incorporated in the porous matrix or in the sample receiving member, if present, such that they dissolve or disperse in the aqueous liquid sample. Alternatively, the developing reagents can be added to the sample before contact with the porous matrix or the porous matrix can be exposed to the developing reagents after the binding reaction has taken place. For example, glycan binding reagents, e.g. lectin, galectin or antibody, may be conjugated with an enzyme (e.g., HRP or alkaline phosphatase) with the intention to react with a color-generating substrate. The conjugate binds to the glycan which is captured by an immobilized agent on the surface, and a substrate that is present in the probe's matrix is used to generate a colored species in the enzyme-catalyzed reaction (the substrate can form e.g. a precipitate or a color).

Coupling of the label to the specific binding reagent can be by covalent bonding, if desired, or by hydrophobic bonding. Such techniques are commonplace in the art, and form no part of the present invention. In the preferred embodiment, where the label is a direct label such as a colored latex particle, hydrophobic bonding or passive adsorption is preferred.

In some embodiments of the invention, the visually detectable label is a “direct label”, attached to one of the specific binding reagents. Exemplary direct labels include gold sols and dye sols, as these are known in the art. These labels can be used to produce an instant analytical result without the need to add further reagents in order to develop a detectable visual signal. They are robust and stable and can therefore be used readily in the device presented herein, which is stored in the dry state. Their release on contact with an aqueous sample can be modulated, for example by the use of soluble glazes.

Preferably, the result of the diagnosis assay should be discernable by eye, and to facilitate this, it is necessary for the visually detectable label to become concentrated in the detection zone. To achieve this, a direct labeling reagent should be transportable easily and rapidly by the developing liquid (the sample's medium). Furthermore, it is preferable that the whole of the developing sample liquid is directed through a comparatively small detection zone in order that the probability of an observable result being obtained in increased.

In some preferred embodiments, the visually detectable label is a colored latex particle of spherical or near-spherical shape and having a maximum diameter of not greater than about 0.5 micron. A preferred size range for such particles is from about 0.05 to about 0.5 microns.

Additional methodologies for visualizing glycans are known in the art, and include analysis of glycans using the bioorthogonal chemical reporter strategy, periodate oxidation, acidic ninhydrin assay, orcinol assay (Bial's test) for visual determination of pentose found in glycans, p-bromoaniline assay, phloroglucinol assay, hexokinase assay, glucose oxidase assay, glucose dehydrogenase assay, D-glucitoldehydrogenase assay, resorcinol assay, and more.

Phenol-sulfuric acid chemistry aimed at generating a color with hexoses and pentoses, found in glycans, can also be used as a visually detectable labels. For a review of this type of labeling, the artisan can turn to, for example, Masuko, T. et al. [“Carbohydrate analysis by a phenol-sulfuric acid method in microplate format”, Analytical Biochemistry, 2005, 339(1), pp. 69-72].

The chemistry of bicinchoninic acid (BCA) as a chromogen, can be used to quantify the amount of copper reduced by the aldehyde present in glycans; the method is sensitive and useful in the range of 1-20 nmol sugar.

Another alternative to visualize glycans is by analysis of free sialic acids from glycoconjugates by thiobarbituric acid. Briefly, periodiate is used under strongly acidic conditions to oxidize N-acetylneuraminic acid (NANA) to β-formylpyruvic acid, which is visible at 549 nm [Crook, M. et al., “Measurement of urine total sialic acid: Comparison of an automated ultraviolet enzymatic method with a colorimetric assay”, British Journal of Biomedical Science, 2002, 59(1), pp. 20-3].

U.S. Pat. No. 5,512,488 provides methods for detacting polysaccharide dissolved in water at alkaline conditions, with the use of Congo Red (sodium diphenyl-bis-α-naphthyl-amine sulfonate) visible at 540 nm, or Crystal Violet, Gentian Violet and Toluidine Blue (Basic Blue or tolonium chloride).

3-Methyl-2-benzothiazolinonehydrazone (MBTH) reacts with the aldehyde moiety of reducing sugars found in glycans, to form a colored adduct, in a reaction that is not interfered by proteins and reducing agents [Gordon E. et al., “Determination of Reducing Sugars with 3-Methyl-2-benzothiazolinonehydrazone”, Anal Biochem, 2001, 305, pp. 287-289].

The purpald reagent (4-amino-3-hydrazino-5-mercapto-1,2,4-triazole, CAS#1750-12-5) is remarkably sensitive and specific for aldehydes found in glycans. The purpald reaction is based on a condensation of formaldehyde with the reagent to form an aminal, which then reacts under aeration to form a purple colored oxidation product. The reaction is sensitive for aldehydes, as ketones are oxidized to an uncoloured product [Jendral, J. A. et al., “Formaldehyde in Alcoholic Beverages: Large Chemical Survey Using Purpald Screening Followed by Chromotropic Acid Spectrophotometry with Multivariate Curve Resolution”, International Journal of Analytical Chemistry, 2011, 2011, 11 pages].

The indicator reagents system (formulation) may also include one or more element that is bound to a magnetic particle, such that immobilization thereof, permanent or temporary, can be achieved by means of a magnetic field. The magnetic particle can be attached to the glycan-based biomarker binding reagent, and/or to the visually detectable label. In some embodiments, the magnetic particle can be the visually detectable label. It is within the scope of the present invention to implement the use of magnetic particles in some embodiments of the present invention, as described, for example, in U.S. Pat. Nos. 4,177,253, 5,320,944, 5,993,740, 5,736,349 and 8,945,469.

The presence or color intensity level of the signal from the label which becomes bound in the detection zone can provide a qualitative or quantitative measurement of analyte in the sample. A plurality of detection zones arranged in series on the porous matrix, through which the aqueous liquid sample can pass progressively, can also be used to provide a quantitative measurement of the analyte, or can be loaded individually with different specific binding agents to provide a multi-analyte test.

In some embodiments where there is a requirement for two or more distinguishable color signals to indicate different events and/or provide control/timing for the diagnostic assay, the device may include more than one type of visually detectable labels. As mentioned above, for the same of clarity, a label that signals the presence of a brain injury glycan-based biomarker is referred to herein as the first visually detectable label, and a label that signals other events or conditions, such as sufficiency of sample amount, sufficiency of elapse time or a positive non-specific binding control, is referred to herein the second visually detectable label. In some embodiments the first and the second labels will be difference chemicals, optionally giving-off different colors, and in some embodiments the first and second labels are the same (identical), which may be attached similarly or differently to the same or different elements in the indicator formulation.

### Alternative Indicator Formulations:

The present invention also encompasses indicator formulations which are not necessarily based on affinity binding of a glycan-based biomarker to an immobilized glycan-based biomarker binding reagent capable of selectively binding to the glycan-based biomarker in a sample. Such formulations can be based on soluble reagents for glycan-based biomarker detection.

For example, in an enzymatic glycan assay embodiment, the analyte (a glycan) in the sample reacts with an enzyme that catalyzes the glycan's decomposition. For example, a hexokinase is an enzyme that phosphorylates hexoses (six-carbon sugars), forming hexose phosphate, which in turn can with another reagent in the indicator formulation to form a substance that gives-off a color. This reagent can be present in the probe or added thereto via a portal, as described herein. Similarly, galactose oxidase is an enzyme that catalyzes the oxidation of D-galactose, and glucose oxidase is an oxido-reductase that catalyses the oxidation of glucose to hydrogen peroxide and D-glucono-δ-lactone; these products of the enzymatic reactions can be detected directly or indirectly.

For example, in a “direct assay” embodiment, the glycan-based biomarker reacts with a chromogen, e.g. a reducing sugar generates reduction of a chromogen thereby affording color development.

In some embodiments based on soluble and diffusible labels, the detection reagent used is glycan-based biomarker binding reagent (e.g., an antibody or lectin) that is conjugated to an enzyme. The glycan biomarker and the detection conjugate are captured on the porous matrix by a pre-immobilized capture reagent (e.g., an antibody or a lectin). Thereafter the unbound conjugate is flushed away via a flushing portal using a flushing solution, and a mixture of substrates and chromogens are added via the same or other portal(s). In some cases the conjugated enzyme is horseradish peroxidase (HRP), the substrate is hydrogen peroxide and the chromogen is 3,3′,5,5′-tetramethylbenzidine (TMB).

In some embodiments based on soluble and diffusible labels, the glycan-based biomarker reacts with an enzyme that is specific for a moiety or a molecular structure present in the glycan-based biomarker. In some cases this moiety can be galactose and the enzyme is galactose oxidase. In the presence of HRP and a chromogen such as Amplex Red (10H-phenoxazine-3,7-diol, 10-acetyl, CAS 119171-73-2) the reaction results in color development (abs max @ 560 nm).

In some embodiments based on soluble and diffusible labels, the glycan-based biomarker reacts directly with a chromogen/dye. In some cases the glycan biomarker can consist of a reducing sugar which in some cases reacts with 3-methyl-2-benzothiazolinonehydrazone (MBTH), developing a colored adduct (see, for example, Sawicki, E. et al. [Anal. Chem., 1961, 33(1), pp 93-96]).

Such formulations can be implemented in a device of any shape and configuration, including a strip and a lollipop configurations, as these are described herein.

### Controls and Timer:

One issue to be reckoned with in a device such as presented herein, is that it takes a little while, after removing the assay device from contact with the liquid sample, for the visible signal to appear (develop). Clearly the user would like to read the result of the assay as soon as possible but, equally, the user requires confidence that sufficient time has elapsed for the proper assay result to have been obtained and that the test is not being read too early, without having to wait an inordinately long period. In order to address this problem, it is known to incorporate a “timer” into an assay device, as described, for example, in U.S. Pat. No. 9,052,311, and EP 0826777, which are incorporated herein by reference in their entirety. These additional ‘timer’ reagents are deposited in a “timer” or “control” zone of the probe and, upon hydration by the sample, interact to produce a color change. In addition, the “timer” or “control” reagents are also said to perform quality-control function. It is generally undesirable if assay devices are exposed to moisture. However, since the timer reagent when hydrated produces a colored product, the timer will reveal if the device has been exposed to moisture, and thus has been tampered with. The “quality control” function indicates whether the device has been exposed to a sufficient or insufficient amount of the sample.

In some embodiments, the probe contains a control formulation, which is associated with a specific zone in the probe, referred to herein a “control zone”. If present, the “control zone” can be designed merely to convey a signal to the user that the device has worked. This signal can be unrelated to the signal indicating the presence of an agent indicative of brain injury in the sample. Preferably, the control zone is located at a different location than the detection zone.

For example, the control zone can be loaded with a control formulation that includes a control binding reagent that will bind to any labeled glycan, to confirm that the sample has permeated and that it contained sufficient analytes therein, and a second visually detectable label. Alternatively, the control formulation in the control zone can include an immobilized analyte which will react with excess labeled reagent, and the purpose of this control zone is to indicate to the user that the test has been completed. A positive control indicator therefore tells the user that the sample has permeated the required distance through the test device. The control binding reagent cab be selected to have binding affinity to the any mobile reagent in the indicator formulation, and a signal that develops from such control formulation will indicate a working device, a sufficient sample, and a timer for completion of the detection process. If only the control zone becomes visually detectable, and the detection zone does not, this scenario indicates that the device is functioning, that the sample acquisition was successful, and that the sample contains no glycan-based biomarker indicative of brain injury.

Alternatively, the control zone can contain an anhydrous reagent that, when moistened, produces a color change or color formation, e.g. anhydrous copper sulfate that will turn blue when moistened by an aqueous sample.

### Exemplary Embodiment of a Device and Mode of Use:

An illustration of an exemplary device, according to some embodiments of the present invention, is designed in the form of a strip, having an elongated flat narrow rectangular shape, wherein one end is used as probe (a detection zone) and the other end is used for holding the strip. According to some embodiments of the present invention, the exemplary device is configured to perform a “yes/no” test for brain injury in a subject by sweeping in the subject's saliva in the mouth or inserting the probe into a container holding the subject's liquid sample, such as urine. Alternatively, the probe can be contacted with the sample by applying/dropping/smearing the liquid sample on the probe. The indicator formulation disposed in/on the probe comprised a labeled and mobile analyte-specific binding reagent (e.g., an antibody or a lectin), an analyte-specific binding reagent (e.g., an antibody or a lectin) immobilized in the detection zone, or chemical compounds or enzymes, which upon presence of the glycan-based biomarker form a color. An exemplary basic device is presented in FIG. 1.

FIG. 1 is a schematic illustration of an exemplary “strip” shaped device, according to some embodiments of the present invention, wherein device 10, having detection zone 11 and handle 12, is dipped into sample 13 not having a glycan-based biomarker, which lead to no coloring of wet detection zone 15, but when dipped into sample 14 having a glycan-based biomarker, wet detection zone 16 changes color.

A strip shaped device is an embodiment in which a wider range of detection chemistries, i.e. diffusible dyes and enzymes that react directly with the glycan-based biomarker, can be used.

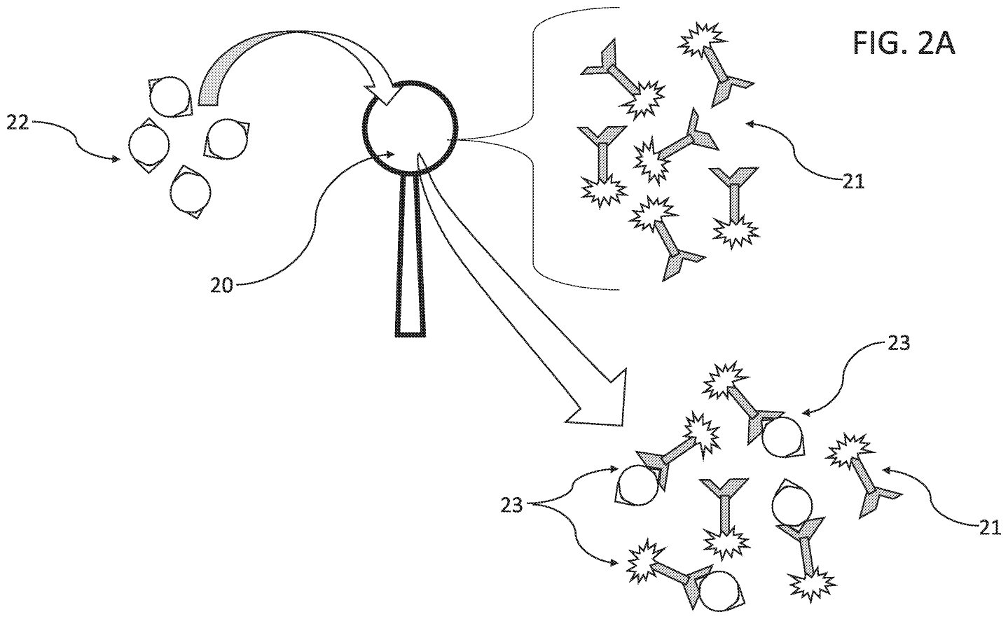

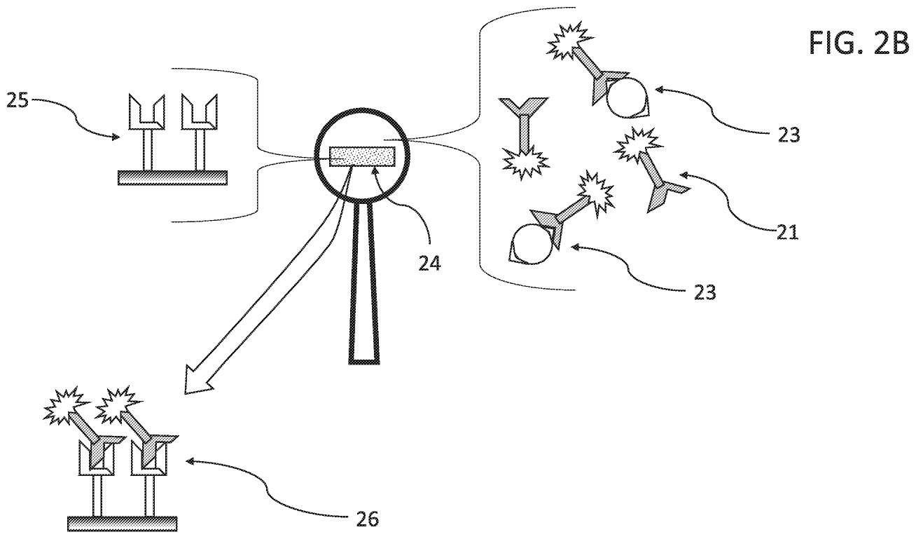

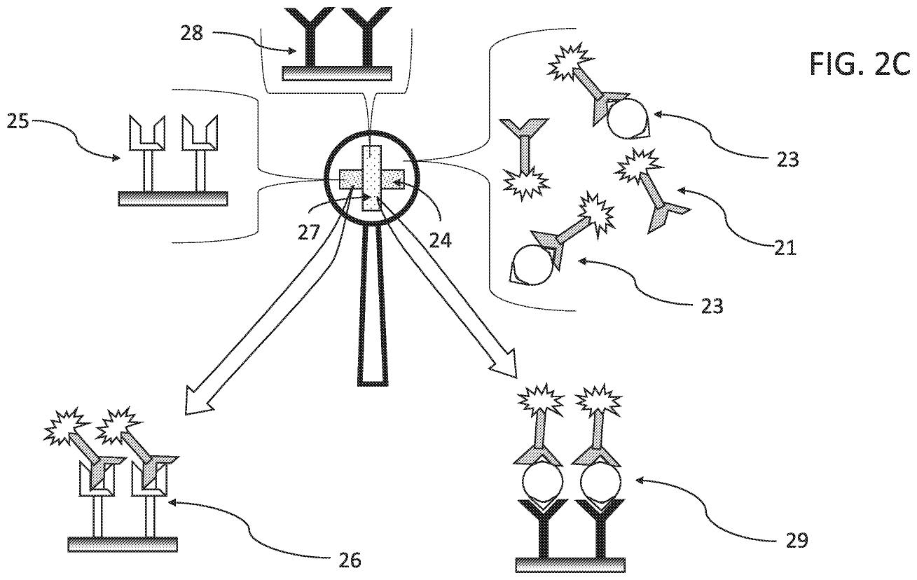

An illustration of another exemplary device, according to some embodiments of the present invention, is designed in the form of a lollipop (round flat probe mounted on a stick handle), having a perpendicular detection zone and a horizontal control zone, relative to the handle. According to some embodiments of the present invention, the exemplary probe is configured to perform a “sandwich” immunoassay to diagnose brain injury in a subject by inserting the probe into the subject's mouth to extract a saliva sample. The indicator formulation disposed in/on the exemplary probe comprised a labeled and mobile analyte-specific binding reagent (e.g., an antibody or a lectin), an analyte-specific binding reagent (e.g., an antibody or a lectin) immobilized in the detection zone, and a nonspecific binding reagent (e.g., an antibody or a lectin) immobilized in the control zone, as presented in FIGS. 2A-C.

FIG. 2A presents a schematic diagram of lollipop device, wherein probe 20 is having mobile labeled antibody (analyte-specific binding reagent) 21 disposed thereon, and when a saliva sample containing glycan-based biomarker (analyte) 22 is contacted with probe 20, mobile labeled antibody-biomarker adduct 23 is formed.

FIG. 2B presents a schematic diagram of the device presented in FIG. 2A, wherein some of mobile labeled antibody 21 was at or has migrated to horizontal control zone 24, in which nonspecific antibody 25 is immobilized on the porous matrix of probe 20, and the binding event is made visible by the label on mobile labeled antibody 21, now immobilized and concentrated in control zone 24 as visibly detectable control complex 26, indicating that the device is functioning properly.

FIG. 2C presents a schematic diagram of the device presented in FIGS. 2A-B, wherein some of mobile labeled antibody-biomarker adduct 23 was at or has migrated to perpendicular detection zone 27, in which biomarker-specific antibody 28 is immobilized on the porous matrix of probe 20, and the binding event is made visible by the label on mobile labeled antibody-biomarker adduct 23, now immobilized and concentrated in detection zone 27 as visibly detectable diagnostic complex 29, indicating that the sample contacted with the device contains glycan-based biomarkers indicative of brain injury.

As can be reckoned from the illustrative example presented in FIGS. 2A-C, a sample containing glycan-based biomarkers indicative of brain injury will evoke the formation of a “plus” symbol at the center of the probe; a sample not containing glycan-based biomarkers indicative of brain injury will evoke the formation of a “minus” symbol at the center of the probe; a misuse of the device with either no sample or insufficient sample will not evoke any visible change at the center of the probe; and an attempt to use of an old, wet, tampered-with device will be prevented by the “minus” sign indicating the device has been moist prior to use.

An illustration of another exemplary device, according to some embodiments of the present invention, is designed to introduce the liquid sample into the probe by plunger-driven motion. The handle, in such embodiments, is hollow and tubular and designed on its distal end to connect to a syringe or another tube or to any other form of liquid transfer mean, while the proximal end is tethered to the probe such that the liquid transported through the handle will soak the porous matrix therein.

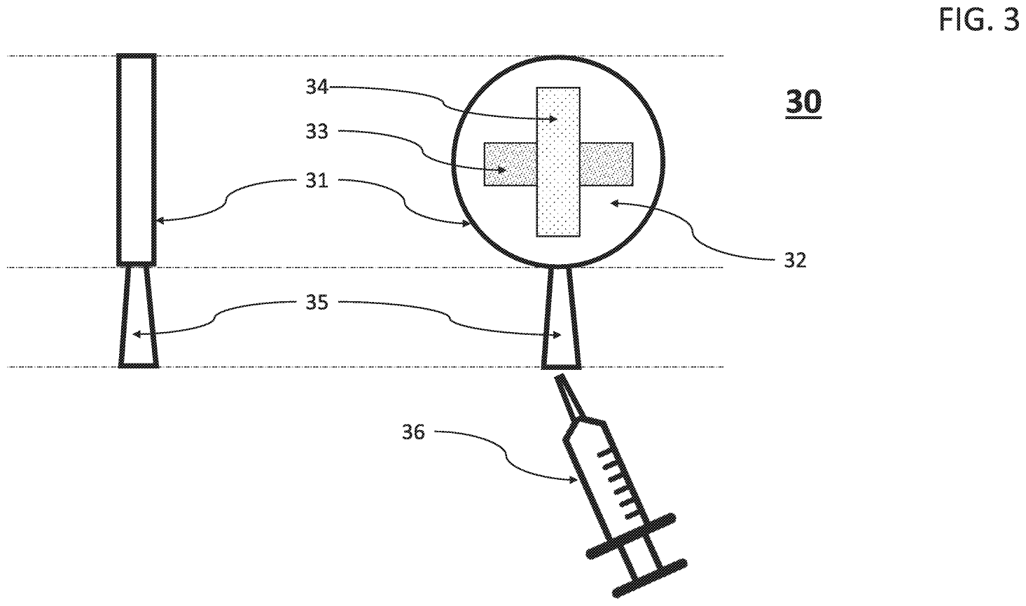

FIG. 3 presents a schematic illustration of a device, according to some embodiments of the present invention, wherein device 30 is having probe 31 comprising porous matrix 32 in which control zone 33 and detection zone 34 form a “plus” sign and handle 35 is a rigid hollow tube designed connect to the tip of generic syringe 36 and transfer the liquid sample to probe 31.

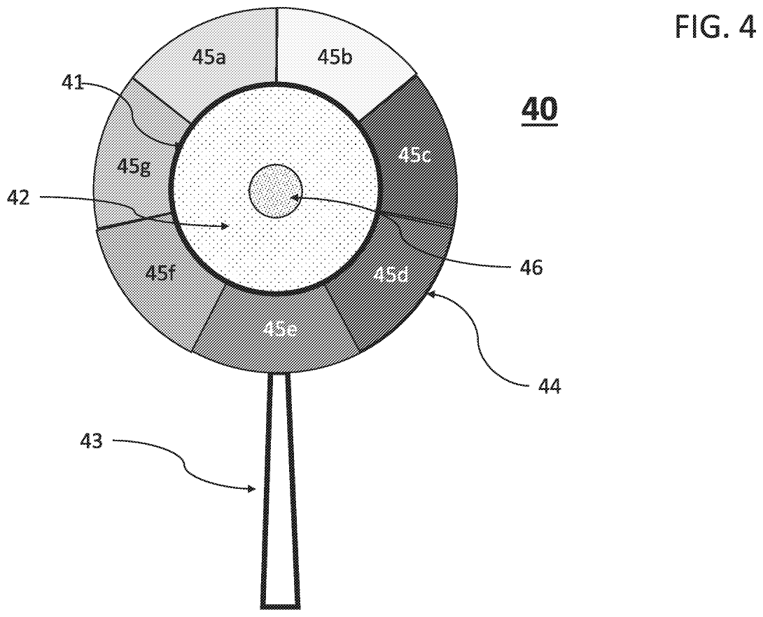

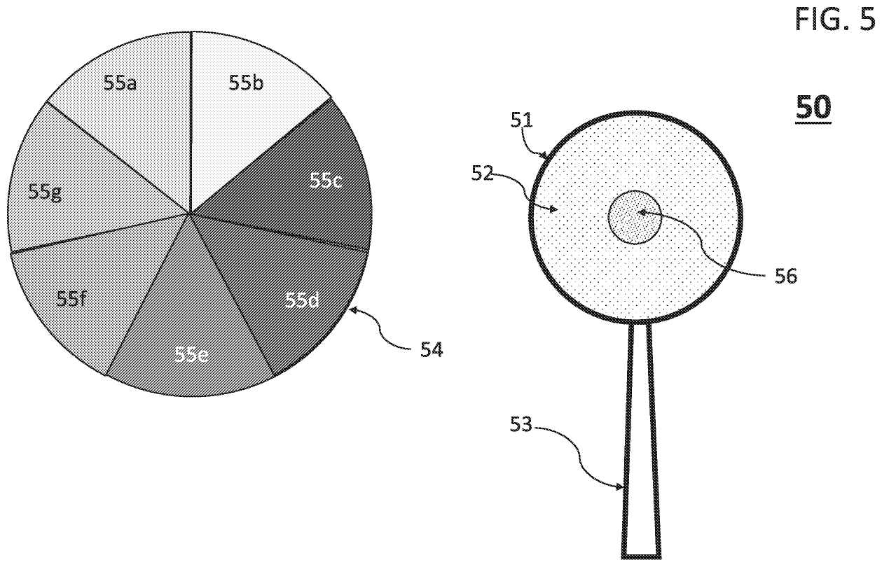

Another illustration of a device, according to some embodiments of the present invention, is a “lollipop” configuration, wherein the probe is placed in a frame that is used also as a gauge for estimating the color intensity level in the probe. In such embodiments, the frame is decorated with a color intensity gauge comprising a plurality of areas arranged radially around the opening that houses the probe. Each of the areas is having a color intensity level representing a concentration level of the glycan-based biomarker in the sample, which is for a visual comparison of a color intensity level in the probe, thereby providing a direct visual determination of a concentration level of the glycan-based biomarker in the sample. FIG. 4 presents a device having an integrated gauge frame, and FIG. 5 presents a device where the gauge is a separate part thereof.

FIG. 4, presents a schematic illustration of a device, according to some embodiments of the present invention, wherein device 40 includes probe 41 that comprises indicator formulation 42, and housed within frame 44, mounted on handle 43, whereas the plurality of areas 45a-g are arranged radially around the opening in frame 44, and control zone 46 is positioned at the center of probe 41.

FIG. 5, presents a schematic illustration of a device, according to some embodiments of the present invention, wherein device 50 includes probe 51 that comprises indicator formulation 52 and control zone 56 is positioned at the center of probe 51, mounted on handle 53, and separate gauge 54 having a plurality of areas 55a-g.

### Method of Diagnosing Brain Injury in a Subject:

In one aspect thereof, the present invention also provides a diagnostic method in which a device as set forth in the foregoing is used to determine a brain injury in a subject. The method is carried out by contacting the device with an aqueous liquid sample suspected of containing the analyte, such that the probe in soaked with the sample that reaches the detection zone, and the control zone if present, through the solid phase porous matrix, and the presence of the analyte in the sample is determined by observing the extent (if any) to which the detection zone changes color.

Thus, according to an aspect of some embodiments of the present invention, there is provided a non-invasive method for diagnosing brain injury in a subject, which is carried out by:

contacting the probe in the device described herein with a sample extracted from the subject in a non-invasive manner;

assessing a visible change in the color of the control zone, if present; and

determining brain injury in a subject according to a color change in the detection zone in the probe,

wherein the change in said color is effected by the binding event of the glycan-based biomarker to the glycan-based biomarker binding reagent(s), and this color change is indicative of a brain injury in the subject.

In some embodiments, a reaction is initiated by the presence of the biomarker, which causes a change in color in the probe. This method is not necessarily based on affinity binding or on immobilization of any one of the elements in the indicator formulation. For example, in some embodiments, an enzyme specific for the glycan-based biomarker is involved in a conversion reaction in the presence of the biomarker. The enzymatic reaction is coupled to a dye/colorant/chromogen which develops color or change it color (enzymatic activity). Such detection method is particularly suitable for the strip device embodiments described herein.

In some embodiments the sample is saliva or urine. The sample extraction may be effected by inserting the device to the mouth of the subject and wetting the probe with saliva. Alternatively, the sample is urine, and the method is effected by wetting the probe with urine taken from the subject.

Glycan-Based Brain Injury Biomarkers:

As discussed hereinabove, WO/2016/166419 discloses diagnostic and prognostic glycan-based brain injury biomarkers, which may be used e.g. for identifying subjects with severe TBI/ABI, who are at risk of secondary brain injury and therefore require increased surveillance, or subjects with mild TBI/ABI or subclinical brain injury (SCI), who otherwise may remain undiagnosed and untreated. The biomarkers disclosed in WO/2016/166419 may also be applied in cases where there are no external signs of injury or where the injured person, such as a baby or a coma patient, cannot describe the injury. For example, brain injury status includes, without limitation, the presence or absence of brain injury in a subject, the risk of developing brain injury, the stage or severity of brain injury, the progress of brain injury (e.g., progress of brain injury over time) and the effectiveness or response to treatment of brain injury (e.g., clinical follow up and surveillance of brain injury after treatment). Based on this status, further procedures may be indicated, including additional diagnostic tests or therapeutic procedures or regimens.

As used herein, the term “biomarker” refers to a molecule that is detectable in a biological sample obtained from a subject and that is indicative of a brain damage in the subject. Markers of particular interest in the invention include glycan-based biomarkers showing differences in glycosylation between a sample from an individual with a brain damage and a healthy control.

As used herein, the term “glycan-based biomarker” refers to monosaccharides and polysaccharides, i.e. a polymer comprising two or more monosaccharide residues, as well as to a carbohydrate portion of a glycoconjugate, such as glycopeptides and glycoproteins, glycolipid, a peptidoglycan, or a proteoglycan, and any fragment thereof. Glycan-based biomarkers may comprise either homo-polymeric or hetero-polymeric monosaccharide residues, and they may be either linear or branched. As used herein, the terms “glycan”, “polysaccharide” and “carbohydrate” are interchangeable, unless otherwise indicated. Glycan-based biomarkers include but are not limited to carbohydrates, sugars, glycans, monosaccharides and/or polysaccharides, glycoproteins and glycopolymers. These biomarkers may be present in blood plasma or serum after brain injury, in cerebrospinal fluid (CSF) after brain injury, in lymph fluid after brain injury, in urine after brain injury, in saliva after brain injury, in tears after brain injury or in exudate after brain injury.

Glycocalyx is an extracellular polymeric coating surrounding many prokaryotic and eukaryotic cells consisting of glycoproteins, glycolipids, proteoglycans and glycosaminoglycans. The constituents of the glycocalyx play an important role e.g. in the process of cell signaling, virus transfection, and immunity.

The biomarkers are differentially present in unaffected subjects (normal control or non-brain injury) and subjects with brain injury, and, therefore, are useful in aiding in the determination of brain injury status. In certain embodiments of the present invention, the biomarkers are measured in a sample taken from a subject using the methods described herein and compared, for example, to predefined biomarker levels and correlated to brain injury status. In particular embodiments, the measurement(s) may then be compared with a relevant diagnostic amount(s), cut-off(s), or multivariate model scores that distinguish a positive brain injury status from a negative brain injury status. The diagnostic amount(s) represents a measured amount of a biomarker(s) above which or below which a subject is classified as having a particular brain injury status. For example, if the biomarker(s) is/are up-regulated compared to normal during brain injury, then a measured amount(s) above the diagnostic cut-offs(s) provides a diagnosis of brain injury. Alternatively, if the biomarker(s) is/are down-regulated during brain injury, then a measured amount(s) at or below the diagnostic cut-offs(s) provides a diagnosis of non-brain injury. As is well understood in the art, by adjusting the particular diagnostic cut-off(s) used in an assay, one can increase sensitivity or specificity of the diagnostic assay depending on the preference of the diagnostician. In particular embodiments, the particular diagnostic cut-off can be determined, for example, by measuring the amount of biomarkers in a statistically significant number of samples from subjects with the different brain injury statuses, and drawing the cut-off to suit the desired levels of specificity and sensitivity.

An advantage of cerebrospinal fluid biomarkers is that the CSF is in direct contact with the extracellular matrix in the brain and, thus, it mirrors biochemical changes in the brain. For these reasons, the CSF might be considered an optimal source of biomarkers of brain injury. However, given that CSF must be obtained by invasive lumbar puncture, availability of biomarkers of brain damage that can be assayed in blood samples would be beneficial. Serum, plasma, saliva or urine biomarkers are of special importance in especially blast-induced TBI because they are typically associated with military operations with limited access to imaging and other diagnostic tools of hospitals. The combination of physical damage and psychological effects makes blast-induced TBI especially difficult to diagnose. Thus, plasma, serum, saliva or urine biomarkers that can distinguish between the physical and psychological components of the injury would be of special value.

As used herein, the term “brain damage” refers to the destruction or degeneration of brain cells due to one or more internal or external factors. Non-limiting examples of brain damage include traumatic brain injury (TBI), acquired brain injury (ABI), subclinical brain injury (SCI) and neurodegenerative conditions. Non-limiting examples of typical neurodegenerative conditions include Huntington's disease, Parkinson's disease, Alzheimer's disease and Chronic Traumatic Encephalopathy. As used herein, the terms “brain damage” and “brain injury” are interchangeable, unless otherwise indicated.

As used herein, the term “traumatic brain injury” (TBI) refers to brain injury caused by external physical trauma, or by a sudden motion of the head, without a physical contact with or hit to an external object. Non-limiting examples of incidences resulting in TBI include falls, vehicle collisions, sports collisions, and combats. The term includes both mild and severe TBI including closed-head injuries, concussions or contusions and penetrating head injuries.

As used herein, the term “acquired brain injury” (ABI) refers to a brain damage not caused by an external brain injury or a hereditary condition. ABI may occur after birth as a result of complications, a disorder or congenital malady, or it may result from, for instance, stroke, surgery, removal of a brain tumor, infection, chemical and/or toxic poisoning, hypoxia, ischemia, sub-stance abuse, or a combination thereof.

The term “brain injury” also refers to subclinical brain injury, and anoxic-ischemic brain injury. The term “subclinical brain injury” (SCI) refers to brain injury without overt clinical evidence of brain injury. A lack of clinical evidence of brain injury when brain injury actually exists could result from degree of injury, type of injury, level of consciousness and/or medications, particularly sedation and anesthesia.

As used herein, the term “subject” refers to any mammal, including animals and human subjects. Animals include, but are not limited to, pets, farm animals, working animals, sporting animals, show animals, and zoo animals. Non-limiting examples of typical human subjects suffering from or pre-disposed to brain damage, TBI in particular, include babies, infants, children and young adults, particularly male; elderly; athletes, particularly boxers, ice-hockey players, soccer players, football (American) players, and skateboarders; and soldiers. The terms “human subject” and “individual” are interchangeable. Typically, the subject is known to have or suspected of having a brain injury, such as TBI or ABI.

As used herein, the term “diagnosis” means detecting an injury, a disease or a disorder, jointly referred to as a medical condition, or determining the stage or degree of the medical condition. Usually, a diagnosis of a medical condition is based on the evaluation of one or more factors (e.g., biomarkers) and/or symptoms that are indicative of the disease and/or its progress. That is, a diagnosis can be made based on the presence, absence or amount of a factor which is indicative of presence or absence of the medical condition. Each factor or symptom that is considered to be indicative for the diagnosis of a particular medical condition does not need be exclusively related to the particular medical condition, i.e. there may be differential diagnoses that can be inferred from a diagnostic factor or symptom. Likewise, there may be instances where a factor or symptom that is indicative of a particular medical condition is present in an individual that does not have the particular disease. The term “diagnosis” also encompasses determining the therapeutic effect of a drug therapy, or predicting the pattern of response to a drug therapy. The diagnostic methods may be used independently, or in combination with other diagnosing and/or staging methods known in the medical arts for a particular medical condition.

In the context of embodiments of the present invention, the term “diagnosis” refers to the determination of whether or not a subject has a brain damage, such as TBI or ABI. The term is also meant to include instances where the presence of a brain damage is not finally determined but that further diagnostic testing is warranted. In such embodiments, the method is not by itself determinative of the presence or absence of a brain damage in the subject but can indicate that further diagnostic testing is needed or would be beneficial. The methods, therefore, can be combined with one or more other diagnostic methods for the final determination of the presence or absence of a brain damage in the subject. Examples of such other diagnostic methods include, but are not limited to, CT and MRI, and are well known to a person skilled in the art. As used herein, a “final determination” or “final diagnosis” refers to ascertaining the presence or absence of a brain damage in a subject. The final determination or final diagnosis can be the result of any of the methods of the invention which, in some embodiments, can include more than one diagnostic test.

As used herein, the term “comparing” refers to making an assessment of how the proportion, level or cellular localization of one or more biomarkers in a sample from a subject relates to the proportion, level or localization of the corresponding one or more biomarkers in a standard or control sample. For example, “comparing” may refer to assessing whether the proportion, level, or cellular localization of one or more biomarkers in a sample from a subject is the same as, more or less than, or different from the proportion, level, or localization of the corresponding one or more biomarkers in standard or control sample. More specifically, the term may refer to assessing whether the proportion, level, or cellular localization of one or more biomarkers in a sample from a subject is the same as, more or less than, different from or otherwise corresponds (or not) to the proportion, level, or cellular localization of predefined biomarker levels that correspond to, for example, a subject having subclinical brain injury (SCI), not having SCI, is responding to treatment for SCI, is not responding to treatment for SCI, is/is not likely to respond to a particular SCI treatment, or having/not having another disease or condition. In a specific embodiment, the term “comparing” refers to assessing whether the level of one or more biomarkers of the present invention in a sample from a subject is the same as, more or less than, different from other otherwise correspond (or not) to levels of the same biomarkers in a control sample (e.g., predefined levels that correlate to uninfected individuals, standard SCI levels, etc.).

The biomarkers and methods presented herein may be used not only for diagnostic purposes but also for prognosis or predicting the outcome of the brain damage, or monitoring the subject's survival from the brain damage or response to treatment.

The biomarkers and methods presented herein may be used as a clinical end point in clinical trials for treating TBI or ABI, providing the outcome of the brain damage, or monitoring the subject's survival from the brain damage or response to treatment.

In some embodiments of the present invention, the diagnosis or prognosis of a brain damage may comprise determination of the presence or absence of one or more of the present glycan-based biomarkers in a biological sample obtained from a subject whose possible brain damage is to be determined. Multiplexed assays can provide substantially improved diagnostic precision. In a specific embodiment, the present invention provides methods for determining the risk of developing brain injury in a subject. Biomarker percentages, amounts or patterns are characteristic of various risk states, e.g., high, medium or low. The risk of developing brain injury is determined by measuring the relevant biomarkers and then either submitting them to a classification algorithm or comparing them with a reference amount, i.e., a predefined level or pattern of biomarkers that is associated with the particular risk level.

In some embodiments, the present invention provides methods for determining the severity of brain injury in a subject. Each grade or stage of brain injury likely has a characteristic level of a biomarker or relative levels of a set of biomarkers (a pattern). The severity of brain injury is determined by measuring the relevant biomarkers and then either submitting them to a classification algorithm or comparing them with a reference amount, i.e., a predefined level or pattern of biomarkers that is associated with the particular stage.

In some embodiments of the present invention, the diagnosis or prognosis of a brain damage may comprise determination of the amount of one or more glycan-based biomarkers, or the relative amounts thereof as compared to, for example, the amount of each other, one or more other glycan, and/or a known standard. In some embodiments, diagnosis or prognosis of brain damage may be based on relative ratios of glycan-based biomarkers in different body fluids, such as a saliva/urine ratio, or a blood/CSF ratio.

In some embodiments, the amounts or relative ratios of one or more glycan-based biomarker may be compared to a predetermined threshold value which is indicative of the presence or absence of a brain damage or is useful in assessing the progression or regression of the brain damage. Such a comparison to a threshold value may result in a final or non-final diagnosis or a determination in regard to the progression or regression of the brain damage. Statistical methods for determining appropriate threshold values will be readily apparent to those of ordinary skill in the art. The threshold values may have been determined, if necessary, from samples of subjects of the same age, race, gender and/or disease status, etc. The threshold value may originate from a single individual not affected by a brain damage or be a value pooled from more than one such individual.

In some embodiments, glycan-based biomarkers may also be detected and/or quantified with the use of lectins. Lectins are a well-known family of carbohydrate-binding proteins, i.e. macromolecules that are highly specific for given glycans on the basis of their sugar moiety structures and sequences. Lectins can be classified into distinct groups according to their carbohydrate specificity including, but not limited to, fucose-specific, mannose specific, N-acetylglucosamine-specific, and galactose/N-acetylglucosamine-specific lectins. It is noted that different sample types may exhibit different profiles of lectin-binding glycan biomarkers. Accordingly, lectins capable of identifying subjects with brain injury may be used in either individually or in any combination thereof.

In some embodiments, glycan-based biomarkers may also be detected and/or quantified with the use of galectins, the most widely expressed class of lectins in all organisms. Galectins are a family of proteins defined by their binding specificity for β-galactoside sugars, such as N-acetyllactosamine (Gai i-3GlcNAc or Gai i-4GlcNAc), which can be bound to proteins by either N-linked or O-linked glycosylation. They are also termed S-type lectins due to their dependency on disulfide bonds for stability and carbohydrate binding. Among fifteen galectins discovered in mammals, only galectin-1, -2, -3, -4, -7, -8, -9, -10, -12 and -13 have been identified in humans, to date. As used herein, “galectins” are encompassed by the term “lectins”, unless otherwise indicated.

### Biomarker Analysis:

Standard techniques of protein microarray technology can be applied to analyze the glycan-based biomarkers. In such microarrays, lectins are immobilized on a solid support, such as a slide, in a high spatial density. Each lectin may be arrayed at several concentrations and in replicates on each slide. The concentration ranges may be tailored for each of the lectins and calibrated to provide a linear response within the same range, regardless of the affinity of the lectin. A sample of intact glycan-based biomarkers is applied to the array, and its binding pattern is detected by a label, such as a fluorescent label, a radioactive label, or a chemiluminescent label, which is placed either on the biomarker itself or on the lectin directed toward the carbohydrate moieties of the biomarker. Streptavidin may be used for detecting biotinylated samples. Also, “sandwich” based methods which utilize antibody detection may be employed, as is apparent to those with ordinary skill in the art.

Suitable microarray substrates include, but are not limited to, glass, silica, aluminosilicates, borosilicates, metal oxides such as alumina and nickel oxide, gold, various clays, nitrocellulose or nylon. In some embodiments a glass substrate is preferred. In other embodiments, the substrate may be coated with a compound to enhance binding of the lectin to the substrate. In some further embodiments, lectins have been arrayed on a nitrocellulose membrane-coated glass slide. In some still further embodiments, one or more control lectins are also attached to the substrate.

In some embodiments, a commercially available lectin array, which encompasses one standard glass slide, which is spotted with 8 wells of identical lectin arrays, may be employed. Each lectin, together with the positive controls is arrayed in duplicate. The slide comes with an 8-well removable gasket which allows for the process of 8 samples using one slide. Four-slide slides can be nested into a tray, which matches a standard microplate and allows for automated robotic high throughput process of 64 arrays simultaneously. Unlike other conventional methods, e.g., liquid chromatography and mass spectrometry, lectin microarrays enable rapid and high-sensitivity profiling of complex glycan features without the need for liberation of glycans. Target samples include an extensive range of glycoconjugates involved in cells, tissues, body fluids, as well as synthetic glycans and their mimics. Various procedures for rapid differential glycan profiling have been developed for glycan-related biomarkers and are commercially available.

In one embodiment, the present invention provides methods for determining the course and prognosis of brain injury in a subject. Brain injury course refers to changes in brain injury status over time, including brain injury progression (worsening) and brain injury regression (improvement). Over time, the amount or relative amount (e.g., the pattern) of the biomarkers changes. For example, biomarker “X” may be increased with brain injury, while biomarker “Y” may be decreased with brain injury. Therefore, the trend of these biomarkers, either increased or decreased over time toward brain injury or non-brain injury indicates the course of the condition. Accordingly, this method involves measuring the level of one or more biomarkers in a subject at least two different time points, e.g., a first time and a second time, and comparing the change, if any. The course of brain injury is determined based on these comparisons.

In some embodiments of the present invention, methods for determining the therapeutic efficacy of a pharmaceutical drug are provides. These methods are useful in performing clinical trials of the drug, as well as monitoring the progress of a subject on the drug. Therapy or clinical trials involve administering the drug in a particular regimen. The regimen may involve a single dose of the drug or multiple doses of the drug over time. The doctor or clinical researcher monitors the effect of the drug on the patient or subject over the course of administration. If the drug has a pharmacological impact on the condition, the amounts or relative amounts (e.g., the pattern or profile) of one or more of the biomarkers of the present invention may change toward a non-brain injury profile. Therefore, one can follow the course of one or more biomarkers in the subject during the course of treatment. Accordingly, this method involves measuring one or more biomarkers in a subject receiving drug therapy, and correlating the biomarker levels with the brain injury status of the subject (e.g., by comparison to predefined levels of the biomarkers that correspond to different brain injury statuses). One embodiment of this method involves determining the levels of one or more biomarkers in minimum at two different time points during a course of drug therapy, e.g., a first time and a second time, and comparing the change in levels of the biomarkers, if any. For example, the levels of one or more biomarkers can be measured before and after drug administration or at two different time points during drug administration. The effect of therapy is determined based on these comparisons. If a treatment is effective, then the one or more biomarkers will trend toward normal, while if treatment is ineffective, the one or more biomarkers will trend toward brain injury indications.

Suitable methods for use in detecting or analyzing glycan-based biomarkers include, but are not limited to, Biocore studies, mass spectrometry, electrophoresis, nuclear magnetic resonance (NMR), chromatographic methods or a combination thereof. Specifically, the mass spectrometric method can be, for example, LC-MS, LC-MS/MS, MALDI-MS, MALDI-TOF, TANDEM-MS, FTMS, multiple reaction monitoring (MRM), quantitative MRM, or Label-free binding analysis. Examples of mass spectrometers are time-of-f light, magnetic sector, quadrupole filter, ion trap, ion cyclotron resonance, electrostatic sector analyser, hybrids or combinations of the foregoing, and the like. In yet another embodiment, mass spectrometry can be combined with another appropriate method(s) as may be contemplated by one of ordinary skill in the art. In another embodiment, the mass spectrometric technique is multiple reaction monitoring (MRM) or quantitative MRM. The electrophoretic method can be, for example, capillary electrophoresis (CE) or isoelectric focusing (IEF), and the chromatographic methods can be, for example, HPLC, chromatofocusing, or ion exchange chromatography.

In some embodiments, detecting, measuring and/or analyzing glycan-based biomarkers in a sample may be carried out by any appropriate enzyme assay available in the art. Such assays include, but are not limited to, galactose oxidase assays.

In some further embodiments, one or more different kinds of binding assays may be used for detecting, measuring and/or analyzing the present glycan-based biomarkers. For instance, a competitive lectin/galectin mode may be employed, wherein a pre-labelled glycan competes with a glycan from a sample to be analyzed for a limited number of binding sites offered by the lectin/galectin. Alternatively or in addition, said binding assay may be carried out in a sandwich mode, wherein one lectin/galectin is used to bind a glycan contained in or derived from a sample to be analyzed from one side, and another lectin/galectin, conjugated with a detectable label, binds to the other side of the glycan or the glycan-lectin/galectin complex formed.

In some embodiments, the biomarkers of the present invention can be detected and/or measured by immunoassays, either in a competitive or sandwich mode. Those skilled in the art know how to carry out such immunoassays. Furthermore, antibodies suitable for this purpose are available commercially. Further suitable antibodies may be produced by methods well known in the art.

In some embodiments, a combination of a lectin/galectin assay and an immunoassay may be employed for detecting, measuring and/or analyzing the present biomarkers in a sample taken from a subject. For this purpose, both a capture reagent and a detection reagent are required. Said capture reagent may be a lectin or a galectin, while said detection reagent may be a detectably labelled antibody, or vice versa.

The present invention also contemplates traditional immunoassays including, for example, sandwich immunoassays such as ELISA or fluorescence-based immunoassays, as well as other enzyme immunoassays. In a SELDI-based immunoassay, a bio specific capture reagent for the biomarker is attached to the surface of an MS probe, such as a pre-activated lectin chip array. The biomarker is then specifically captured on the biochip through this reagent, and the captured biomarker is detected by mass spectrometry.

As is readily understood by those skilled in the art, more than one type of lectins/galectins and/or more than one type of antibodies may be used in the binding assays set forth above. In other words, several different lectins/galectins and antibodies may be used in a reaction to enhance the binding affinity or specificity. Furthermore, multiple different reactions may be carried out simultaneously or sequentially for detecting different glycan-based biomarkers in a sample to be analyzed.

It is also contemplated that glycans or glycan complexes contained in a sample to be analyzed may be immobilized directly to a surface, such as a microplate well, a glass surface (e.g. a slide), a metal surface (e.g. a silver or gold leaf) by opposite charges, by a glue, of by affinity binding, and be subsequently detected, for instance, by a detectably labelled lectin or antibody.

In accordance with the above, molecules suitable for use in detecting glycan-based biomarkers in a sample to be analyzed include, but are not limited to, lectins, galectins, antibodies, and competitive small molecules. Said detection molecules may be visualized, or made otherwise measurable, using for instance conjugated color reagents, labels, or dyes. Enzyme labels suitable for this purpose include those that upon addition of a substrate catalyze a reaction leading to a measurable change in color, in luminescence, or in production of a precipitate. Non-limiting examples of such enzyme labels include horseradish peroxidase (HRP) and alkaline phosphatase (AP). Photoluminescent labels, including fluorescent dyes (prompt), lanthanide chelates (for time-resolved fluorescence), and photon upconversion labels may be used for detecting said detection molecules. Furthermore, the detection may be based on bioluminescence and chemiluminescence (as e.g. in luciferin-based detection), or on electrochemiluminescence (with e.g. ruthenium complexes). Also biotin and its derivatives, which enable binding and detection by labeled avidin or labeled streptavidin, as well as various radioactive isotopes may be used for the detection. The detection may also be carried out using beads and particles, including, for example, colored latex particles, colored synthetic polymer particles, colloidal metals such as gold and silver particles, (para)magnetic beads, and fluorophore-dyed particles.

In some embodiments, the biomarkers of the present invention may be detected by means of an electrochemical-luminescent assay developed by Meso Scale Discovery (Gaithersrburg, Md.). Electrochemiluminescence detection uses labels that emit light when electrochemically stimulated. Background signals are minimal because the stimulation mechanism (electricity) is decoupled from the signal (light). Labels are stable, non-radioactive and offer a choice of convenient coupling chemistries.

Furthermore, a sample may also be analyzed by means of a passive or active biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there. Lectin biochips are biochips adapted for the capture of glycans. Many lectin biochips are described in the art.

### Kits:

According to an aspect of embodiments of the present invention, there is provided a kit for non-invasive diagnosis of brain injury in a subject, which can be carried out by any layman at any location and facility without the need for special training, procedures or machinery.

In some embodiment, the kit comprises the device described herein. In some embodiment, the kit further comprises instructions for use of the device and for understanding the various visual signals obtained as a result of using the device. In some embodiment, the kit further comprises a gauge for assessing the concentration of glycan-based biomarkers in the sample.

In some embodiments the kit can be used to determine the presence or absence of, or to measure the levels of one or more glycan-based biomarker. In some embodiments, the kit comprises a package containing one or more glycan-based biomarker binding reagent, such as a lectin or an antibody which selectively binds to one or more glycan-based biomarker, and a control for comparing to a measured value of binding. In some embodiments, the control is a threshold value for comparing to the measured value. The kit can also include a visually detectable label.

According to some embodiments of the present invention, the kit may further include a device, a series of pre-measured (concentration and volume) liquids in separate reservoirs, and a mean to connect each of the reservoirs to the device so as to allow the contents of the reservoir to contact the probe. In some embodiments, the reservoirs are in the form of a plunger/barrel type (e.g., a syringe) which can connect directly to the probe via one of the portals described hereinabove. In some embodiments, the syringes are pre-filled and affixed to the device. In some embodiments, the kit also includes a protective sheath in the form of a plastic or metal container, which can also serve as a sample dipping container, for example, when testing urine. The device can be provided to the user in the protective sheath as a form of packaging that can be used for sample collection and contacting (e.g., dipping).

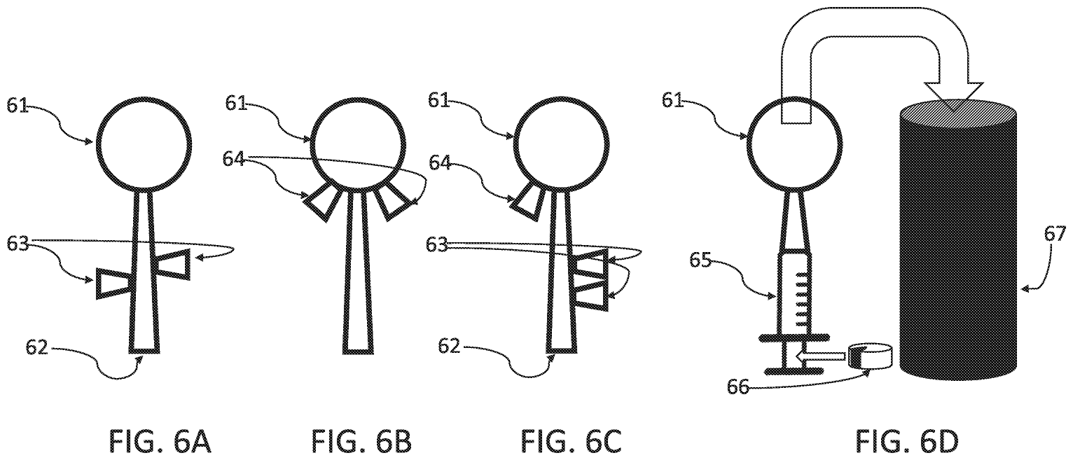

FIGS. 6A-D present schematic illustrations of some embodiments of the present invention, wherein FIG. 6A shows a device having probe 61 in direct communication with handle portal 62 and additional portals 63 branching off from handle portal 62, FIG. 6B shows a device having probe 61 and two portals 64 in direct communication with probe 61, FIG. 6C shows a device having portal 64 in direct communication with probe 61 and additional portals 63 branching off from handle portal 62, and FIG. 6D shows a device having probe 61 in direct communication with handle reservoir 65 in the form of a syringe that is secured from accidental or premature ejection of its content by plunger stopper 66 as part of a kit and protective sheath 67 that can also serve as a sample dipping container as part of a kit.

The kit for qualifying brain injury status may be provided as an immuno-chromatography strip comprising a membrane on which the antibodies are immobilized, and a means for detecting the biomarker(s). The kit may comprise a plastic plate on which a sample application pad, on which antibody bands and a secondary antibody band are immobilized and an absorbent pad are positioned in a serial manner, so as to keep continuous capillary flow of blood serum.

In some embodiments, a subject can be diagnosed by adding blood, plasma or serum from the subject to the kit and detecting the relevant biomarkers conjugated with antibodies, specifically, by a method which comprises the steps of: (i) collecting blood, plasma or serum from the subject; (ii) separating blood serum from the subject's blood; (iii) adding the blood plasma or serum from subject to a diagnostic kit; and, (iv) detecting the biomarkers conjugated with antibodies. In this method, the antibodies are brought into contact with the subject's blood. If the biomarkers are present in the sample, the antibodies will bind to the sample, or a portion thereof. In other kit and diagnostic embodiments, blood, plasma or serum need not be collected from the subject (i.e., it is already collected). Moreover, in other embodiments, the sample may comprise a tissue sample or a (non-invasive) clinical sample such as saliva, urine or other body fluids as described herein.

The kit can also comprise a washing solution or instructions for making a washing solution, in which the combination of the capture reagents and the washing solution allows capture of the biomarkers on the solid support or column for subsequent detection by, e.g., antibodies or mass spectrometry. In a further embodiment, a kit can comprise instructions for suitable operational parameters in the form of a label or separate insert. For example, the instructions may inform a consumer about how to collect the sample, how to wash the probe or the particular biomarkers to be detected, etc. In yet another embodiment, the kit can comprise one or more containers with biomarker samples, to be used as standard(s) for calibration.

As is apparent to a skilled person, the present lectin array kit can be used with either a label-based method or as a sandwich-based method. In one embodiment, the label based method is used for biotinylated samples containing proteoglycans and glycoproteins for direct detection on the array via a Cy3 equivalent dye-conjugated Biotin-Streptavidin complex. In some embodiments, a sandwich-based method is used for antibody detection of glycocalyx elements (glycolipids, glycoproteins, etc.) captured on the array. Labelled re-porter antibodies specific for the glycocalyx elements of interest may be provided in the kit or supplied by the user of the kit. An example protocol for this procedure with a general “Antibody Cocktail” may be included in a user manual. In some non-limiting embodiments, specific antibody concentrations and conditions may need to be determined by the end user.

In some embodiments, the biomarker detection kit comprises HRP protein and a fluorescent light may be employed in order to detect the biomarker in a body fluid and to indicate the quantity of the biomarker in percentage. This may be incorporated into a portable application that indicates the severity of brain damage on a scale comprising, but not limited to, none, mild, moderate and severe. In another embodiment, an analogous yes/no reply is received. These examples do not exclude other possible embodiments.

In some embodiments, the present invention provides use of at least one antibody in a kit or in a device to detect brain damage, where the antibody may be a polyclonal or a monoclonal antibody of any species, or a fragment thereof, either enzymatically cleaved or recombinantly produced, or a humanized antibody, and where the antibody recognizes and binds glycan, glycoprotein, peptidoglycan, proteoglycan, glycolipid, protein, small molecule, lectin, or antibody of another species (generally ‘antigens’).

An antibody may be used, for instance, as:

i) a capture reagent, wherein the antibody is immobilized on a solid substrate to bind its antigen from a sample medium;

ii) an antibody that is immobilized on a solid substrate to bind an analyte-specific capture reagent (for example lectin) so that the bound agent (lectin) is able to capture the analyte (glycan) from a sample;

iii) a primary detection reagent, wherein an antibody conjugated to any label (labeled antibody) recognizes and binds directly an antigen;

iv) a secondary detection reagent, wherein a labelled antibody recognizes and binds a primary detection reagent that is bound to the analyte. For example, a labeled antibody binds to a lectin that has bound to its cognate glycan, or a labeled antibody from one species (e.g. goat) that recognizes and binds an antibody of another species (e.g. mouse) which has bound its antigen;

v) an antibody for recognizing and binding a non-glycan part of a glycan-containing molecule, e.g. a glycoprotein, where the glycoprotein or a fragment thereof is first bound to e.g. lectin via its glycan moiety and then is recognized and bound by an antibody that is specific to the peptide part of the molecule; or

vi) antibody for use in immunoblotting assays.

The kit may also comprise a combination of antibodies for different purposes.

All embodiments, details, advantages, and the like of the present device also apply to a device for use in different aspects and embodiments of the present invention. Also, all embodiments, details, advantages, and the like of the present methods apply to the present kit, and vice versa. In particular, one or more compounds, compositions, or reagents disclosed as suitable for carrying out the present methods may be comprised in the present kit. Likewise, anything disclosed with reference to the kit, apply to the present methods as well.

Without further elaboration, it is believed that one skilled in the art, using the preceding description, can utilize the present invention to the fullest extent.

Non-limiting examples of advantages associated with the present glycan-based biomarkers include that they are brain-tissue specific, able to cross the blood-brain barrier into the bloodstream within minutes of injury, and can be detected using a point-of-care blood test or other body fluids. Furthermore, the biomarkers may either increase or decrease following the injury, but nevertheless they are in correlation with the severity of the injury. Preferably, the present biomarkers may correlate with injury magnitude, survivability, and/or neurologic outcome, or they may be indicative of the extent of neuronal and glial cell loss, axonal, and vascular damage. The present biomarkers can significantly add to the current diagnostic palette for brain damage.

It is expected that during the life of a patent maturing from this application many relevant saliva-based brain injury diagnosis devices will be developed and the scope of the term saliva-based brain injury diagnosis device is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the phrases “substantially devoid of” and/or “essentially devoid of” in the context of a certain substance, refer to a composition that is totally devoid of this substance or includes less than about 5, 1, 0.5 or 0.1 percent of the substance by total weight or volume of the composition. Alternatively, the phrases “substantially devoid of” and/or “essentially devoid of” in the context of a process, a method, a property or a characteristic, refer to a process, a composition, a structure or an article that is totally devoid of a certain process/method step, or a certain property or a certain characteristic, or a process/method wherein the certain process/method step is effected at less than about 5, 1, 0.5 or 0.1 percent compared to a given standard process/method, or property or a characteristic characterized by less than about 5, 1, 0.5 or 0.1 percent of the property or characteristic, compared to a given standard.

The term “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The words “optionally” or “alternatively” are used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the terms “process” and “method” refer to manners, means, techniques and procedures for accomplishing a given task including, but not limited to, those manners, means, techniques and procedures either known to, or readily developed from known manners, means, techniques and procedures by practitioners of the chemical, material, mechanical, computational and digital arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental and/or calculated support in the following examples.

### EXAMPLES

Reference is now made to the following examples, which together with the above descriptions illustrate some embodiments of the invention in a non-limiting fashion.

### Example 1

### Selection of Porous Matrix Material

Representative examples of porous matrix materials include paper, nitrocellulose and nylon membranes. Essential features of the material are its ability to bind protein; speed of liquid conduction; and, if necessary after pre-treatment, its ability to allow the passage of labeled binding reagents therethrough. In embodiments using direct label, it may be desirable for the material to allow flow of particles of size up to a few microns (usually less than 0.5 μm). Examples of flow rates obtained with various materials are presented in Table 1 below, showing the time in minutes to flow through 45 mm of material.

TABLE 1

| | Pore size | Time |
|---|---|---|
| Material type | [μm] | [minutes] |
| Whatman ®'s chromatography paper | | |
| (Schleicher & Schuell ®) | 3 | 3.40 |
| unbacked nitrocellulose sheet | 5 | 3.30 |
| | 8 | 3.00 |
| | 12 | 2.20 |
| backed polyester sheet | 8 | 3.40 |
| Whatman ®'s Nitrocellulose sheet | 5 | 19.20 |
| Pall ®'s Immunodyne ® (nylon) | 3 | 4.00 |
| | 5 | 3.20 |

The travel rate of a test procedure will be determined by the flow rate of the material employed and while any of the above materials can be used some will give faster tests than others.

Nitrocellulose is advantageous of requiring no activation and will immobilize proteins strongly by absorption. Immunodyne® is pre-activated and requires no chemical treatment. Papers, such as Whatman®'s 3MM, require chemical activation with for example carbonyldiimidazole in order to successfully immobilize proteins.

### Example 2

### Preparation of Labels

A selection of labels which may be used are described below. This list is not exhaustive and it is noted that other labeling methodologies and technologies are contemplated within the scope of the present invention.

Gold Sol/Colloid Preparation:

Gold sols may be prepared for use in immunoassay from commercially-available colloidal gold, and an antibody preparation. Metallic sol labels are described, for example, in European patent specification No, EP 7654.

For example, colloidal gold G20 (20 nm particle size, supplied by Janssen Life Sciences Products) is adjusted to pH 7 with 0.22 μm filtered 0.1 M K₂CO₃, and 20 ml is added to a clean glass beaker. 200 μl of antibody, prepared in 2 mM borax buffer pH 9 at 1 mg/ml, and 0.22 μm filtered, is added to the gold sol, and the mixture stirred continuously for two minutes. 0.1M K₂CO₃ is used to adjust the pH of the antibody gold sol mixture to 9, and 2 ml of 10% (w/v) BSA is added.

The antibody-gold is purified in a series of three centrifugation steps at 12000 g, 30 minutes, and 4° C., with only the loose part of the pellet being resuspended for further use. The final pellet is resuspended in 1% (w/v) BSA in 20 mM Tris, 150 mM NaCl pH 8.2.

Dye Sol Preparation:

Dye sols (see, for example, European patent specification No. EP 32270) may be prepared from commercially-available hydrophobic dyestuffs such as Foron Blue SRP (Sandoz) and Resolin Blue BBLS (Bayer). For example, fifty grams of dye is dispersed in 1 liter of distilled water by mixing on a magnetic stirrer for 2-3 minutes. Fractionation of the dye dispersion can be performed by an initial centrifugation step at 1500 g for 10 minutes at room temperature to remove larger sol particles as a solid pellet, with the supernatant suspension being retained for further centrifugation.

The suspension is centrifuged at 3000 g for 10 minutes at room temperature, the supernatant being discarded and the pellet resuspended in 500 ml distilled water. This procedure is repeated a further three times, with the final pellet being resuspended in 100 ml distilled water.

The spectra of dye sols prepared as described above can be measured, giving lambda-max values of approximately 657 nm for Foron Blue, and 690 nm for Resolin Blue. The absorbance at lambda-max, for 1 cm path length, is used as an arbitrary measure of the dye sol concentration.

Colored Particles:

Latex (polymer) particles for use in immunoassays are commercially available. These can be based on a range of synthetic polymers, such as polystyrene, polyvinyltoluene, polystyrene-acrylic acid and polyacrolein. The monomers used are normally water-insoluble, and are emulsified in aqueous surfactant so that monomer micellae are formed, which are then induced to polymerize by the addition of initiator to the emulsion. Substantially spherical polymer particles are produced.

Colored latex particles can be produced either by incorporating a suitable dye, such as anthraquinone (yellow), in the emulsion before polymerization, or by coloring the pre-formed particles. In the latter route, the dye should be dissolved in a water-immiscible solvent, such a chloroform, which is then added to an aqueous suspension of the latex particles. The particles take up the non-aqueous solvent and the dye, and can then be dried. Preferably such latex particles have a maximum dimension of less than about 0.5 micron.

Colored latex particles may be sensitized with protein, and in particular antibody or lectin, to provide selective binding reagents as described in the foregoing. For example, polystyrene beads of about 0.3 micron diameter, (supplied by Polymer Laboratories) may be sensitized with an anti-glycan-based biomarker antibody, in the process described below:

0.5 ml (12.5 mg solids) of suspension is diluted with 1 ml of 0.1M borate buffer pH 8.5 in an Eppendorf vial. These particles are washed four times in borate buffer, each wash consisting of centrifugation for 3 minutes at 13000 rpm in an MSE microcentrifuge at room temperature. The final pellet is resuspended in 1 ml borate buffer, mixed with 300 μg of anti-glycan-based biomarker antibody, and the suspension is rotated end-over-end for 16-20 hours at room temperature. The antibody-latex suspension is centrifuged for 5 minutes at 13000 rpm, the supernatant is discarded and the pellet resuspended in 1.5 ml borate buffer containing 0.5 mg bovine serum albumin. Following rotation end-over-end for 30 minutes at room temperature, the suspension is washed three times in 5 mg/ml BSA in phosphate buffered saline pH7.2, by centrifugation at 13000 rpm for 5 minutes. The pellet is resuspended in 5 mg/ml BSA/5% (w/v) glycerol in phosphate buffered saline pH 7.2 and stored at 4° C. until used.

Anti-Glycan-Based Biomarker Antibody-Dye Sol Preparation:

Protein may be coupled to dye sol in a process involving passive adsorption. The protein may, for example, be a lectin or an antibody preparation such as anti-glycan-based biomarker antibody prepared in phosphate buffered saline pH 7.4 at 2 mg/ml. A reaction mixture is prepared which contains 100 μl antibody solution, 2 ml dye sol, 2 ml 0.1M phosphate buffer pH 5.8 and 15.9 ml distilled water. After gentle mixing of this solution, the preparation is left for fifteen minutes at room temperature. Excess binding sites may be blocked by the addition of, for example, bovine serum albumin: 4 ml of 150 mg/ml BSA in 5 mM NaCl pH 7.4 is added to the reaction mixture, and after 15 minutes incubation at room temperature, the solution is centrifuged at 3000 g for 10 minutes, and the pellet resuspended in 10 ml of 0.25% (w/v dextran/0.5% w/v lactose in 0.04 M phosphate buffer). This antibody-dye sol conjugate is best stored in a freeze dried form.

### Example 3

### Preparation of a Strip Device

In an exemplary embodiment of the present invention, the device can be formed in the shape of a strip, as depicted in FIG. 1.

Zonal Impregnation of Liquid-Conductive Porous Matrix Materials:

Liquid-conducting porous matrix material with a restricted zone of immobilized protein, particularly antibody or lectin, can be prepared for example as follows:

A rectangular sheet of e.g., Schleicher & Schuell backed 8 μm nitrocellulose paper measuring 10 cm in length and 1 cm in width may have a detection zone formed upon it by applying an area of material about 1 cm long at one end of the strip. The material can, for example, be a suitably selected antibody preparation, prepared in phosphate buffered saline pH 7.4 at 2 mg/ml, suitable for a labeled) lectin in a sandwich format. This solution can be deposited by means of a microprocessor-controlled microsyringe, which delivers precise volumes of reagent through a nozzle, preferably 2 mm diameter. When the applied material is been allowed to dry for 1 hour at room temperature, excess binding sites on the nitrocellulose are blocked with an inert compound such as polyvinyl alcohol (1% w/v in 20 mM Tris pH 7.4) for 30 minutes at room temperature, and sheets are thoroughly rinsed with distilled water prior to drying for 30 minutes at 30° C.

In one embodiment, the liquid conductive porous matrix material can be prepared in bulk of wide format sheets, and then be cut up into numerous strips 10 cm in length and 1 cm in width, each strip carrying a detection zone of the immobilized antibody to function as an immunosorbent part at it tip. In this example the test strip is used with a liquid label which is mixed with sample. In use, this detection zone in which the immunoassay reactions take place.

### Example 4

### Fetuin and Asialofetuin

In an exemplary embodiment of the present invention is a model of assaying fetuin and asialofetuin. Fetuin is an abundant glycoprotein in fetal serum, and asialofetuin is its asialylated form. Lectins or a lectin that selectively binds to the glycan part of fetuin or asialofetuin is permanently immobilized on solid matrix. Fetuin or asialofetuin in solution is brought into contact with the lectin and the binding reaction is subsequently taking place. Thereafter, the reaction compartment is washed and a labeled conjugate is added. The conjugate binds to the fetuin or asialofetuin that was captured on the surface in the preceding phase.

Alternatively, fetuin or asialofetuin is first contacted with the labeled conjugate to form a complex. Thereafter the complex is brought into contact with the immobilized lectin(s). The conjugate comprises a fetuin-specific or asialofetuin-specific antibody which is coupled to a detectable label. The detectable label is one of those presented in the text, preferably a colloidal/particulate matter which enables visual detection.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1-24. (canceled)

25. A device for diagnosing a brain injury in a subject, comprising:
a probe, said probe comprises a porous matrix; and
an indicator formulation disposed in and/or on said porous matrix and comprises at least one glycan-based biomarker binding reagent for selectively binding to a glycan-based biomarker in a sample, and a first visually detectable label;
wherein:
at least one of said glycan-based biomarker binding reagent and/or said first visually detectable label is immobilized in and/or on a detection zone in said porous matrix;
said glycan-based biomarker is indicative of brain injury;
said first visually detectable label develops a color and becomes visible upon a binding event of said glycan-based biomarker to said glycan-based biomarker binding reagent; and
said binding event is effected by contacting said sample with said probe.

26. The device of claim 25, wherein said glycan-based biomarker binding reagent is a lectin and/or an antibody.

27. The device of claim 25, wherein said first visually detectable label is attached to said glycan-based biomarker binding reagent.

28. The device of claim 25, wherein said probe further comprises a control formulation, said control formulation comprises a control binding reagent and a second visually detectable label, said control binding reagent binds at least one of said glycan-based biomarker binding reagent, a glycan and any complex thereof, and said second visually detectable label becomes visible upon a binding event of said control binding reagent to said glycan-based biomarker binding reagent, said glycan and/or said complex thereof, wherein said control binding reagent and/or said second visually detectable label is immobilized in and/or on a control zone in said porous matrix.

29. The device of claim 25, wherein a change in an intensity level of said color is proportional to a concentration level of said glycan-based biomarker in said sample.

30. The device of claim 25, further comprising a semi-permeable layer disposed over said probe, said layer is permeable to aqueous media and aqueous solutes therein, and is impermeable to particles larger than 0.05 μm.

31. The device of claim 25, further comprising a handle in communication with said probe.

32. The device of claim 31, wherein said handle comprises a tube in direct communication with said probe on a proximal end thereof, and open on a distal end thereof, said tube is for transporting said sample and/or a solution from an external source to said probe.

33. The device of claim 31, further comprising a frame having an opening, and said probe is housed within said opening in the plane of said frame, and said frame is mounted on said handle.

34. The device of claim 33, wherein said frame comprises a color intensity gauge, said gauge comprises a plurality of areas arranged radially around said opening, each of said areas is having a color intensity level representing a concentration level of said glycan-based biomarker in said sample, for a visual comparison of a color intensity level in said probe with a color intensity level in one of said areas in said gauge, thereby providing a direct visual determination of a concentration level of said glycan-based biomarker in said sample.

35. The device of claim 31, wherein said sample is urine, and said handle is a tube configured for effecting said contacting.

36. The device of claim 25, wherein said sample is saliva, and the device is sized and shaped for insertion into the subject's mouth for effecting said contacting.

37. A device for diagnosing a brain injury in a subject, comprising:
a flat round probe, said probe comprises a porous matrix;
an indicator formulation disposed in and/or on a detection zone in said porous matrix and comprises at least one glycan-based biomarker binding reagent for selectively binding to a glycan-based biomarker in a sample, and a first visually detectable label;
a control formulation disposed in and/or on a control zone in said porous matrix and comprises a control binding reagent and a second visually detectable label; and
a handle in communication with said probe,
wherein:
said glycan-based biomarker is indicative of brain injury;
at least one of said glycan-based biomarker binding reagent and/or said first visually detectable label is immobilized in and/or on said detection zone;
said first visually detectable label develops a color and becomes visible upon a binding event of said glycan-based biomarker to said glycan-based biomarker binding reagent;
said control binding reagent binds at least one of said glycan-based biomarker binding reagent, a glycan and any complex thereof;
said control binding reagent and/or said second visually detectable label is immobilized in and/or on said control zone;
said second visually detectable label becomes visible upon a binding event of said control binding reagent to said glycan-based biomarker binding reagent, said glycan and/or said complex thereof; and
said binding event is effected by contacting said sample with said probe.

38. The device of claim 37, wherein said handle comprises a tube in direct communication with said probe on a proximal end thereof, and open on a distal end thereof, said tube is for transporting said sample and/or a solution from an external source to said probe.

39. The device of claim 37, wherein said handle is configured in a shape selected from the group consisting of a syringe tip fitting/adaptor, a stretchable and elastic fitting/adaptor, a screw threaded fitting/adaptor, a piercing needle tip fitting/adaptor, a septum membrane and a butterfly needle fitting/adaptor.

40. The device of claim 37, wherein said control zone and said detection zone are perpendicular to one another and overlap at the center so as to form a cross pattern.

41. A non-invasive method for diagnosing brain injury in a subject, the method comprising:
contacting said probe in the device of claim 25 with said sample;
assessing a visible change in said control zone, if present; and
determining brain injury in a subject according to a color change in said detection zone,
wherein said change in said color is effected by said binding event of said glycan-based biomarker to said glycan-based biomarker binding reagent, and indicative of a brain injury in the subject.

42. The method of claim 41, wherein said sample is saliva or urine.

43. The method of claim 41, wherein said contacting is effected by inserting the device to the mouth of the subject and wetting said probe with saliva.

44. The method of claim 41, wherein said contacting is effected by wetting said probe with urine of the subject.
